(19) United States
(12) Patent Application Publication
(10) Pub. No.: US 2026/0110029 A1
(43) Pub. Date: Apr. 23, 2026

(54) METHOD FOR DETERMINING PERCENTAGE OF BLOOD CELL SUBTYPES IN A BLOOD SPECIMEN

(71) Applicant: TruDiagnostics, Inc., Lexington, KY (US)

(72) Inventors: Andrew E. Teschendorff, Lexington, KY (US); Ryan M. Smith, Lexington, KY (US)

(21) Appl. No.: 18/276,951

(22) PCT Filed: Mar. 28, 2023
(86) PCT No.: PCT/US2023/016583

§ 371 Date: Aug. 11, 2023

(51) Int. Cl. C12Q 1/6881

(52) U.S. Cl. CPC C12Q 1/6881; C12Q 2600/154

(57) ABSTRACT

A method of determining percentages of twelve blood cell subtypes in a blood specimen, includes steps of: (a) obtaining genomic DNA from the blood specimen of a human subject, (b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil, (c) comparing the unmethylated CG loci observed in (b) to unmethylated CG loci observed in genomic DNA collected from a reference group of human individuals and (d) correlating the unmethylated CG loci observed in (b) with the unmethylated CG loci observed in the reference group of human individuals to determine the percentage of the twelve blood cell subtypes in the blood specimen.

### TECHNICAL FIELD

This document relates generally to the construction and validation of a DNA reference matrix focusing upon certain unmethylated cytosine-phosphate-guanine sites/CG loci of various blood cell subtypes allowing the determination of the percentage of those blood cell subtypes in a blood specimen.

### BACKGROUND

Interpretation of epigenome-wide association data generated from bulk-tissue benefits from adjustment of the underlying cellular heterogeneity. While the statistical adjustment for cell-type heterogeneity can be done at different cellular resolutions, the need and impact of performing such adjustments at an ever higher cellular resolution remains underexplored. Here we derive a novel DNA methylation reference for 12 blood cell subtypes. These blood cell-type fractions have novel associations with age and sex. Notably we found that natural-killer and basophil fractions increase with age, whilst the eosinophil fraction does not, except in childhood cancer survivors who have undergone chemotherapy treatment. We also found that monocyte, natural killer, basophil and eosinophil fractions are higher in men compared to women, whilst the reverse is true for T-regulatory and naïve T-cell subsets. Correspondingly, epigenetic clock age acceleration measures associated with sex are often dampened upon adjustment for cell-type heterogeneity. These data support the view that inferring cell-type fractions at high resolution and subsequent adjustment for variations in these fractions is necessary for a more complete interpretation of DNA methylation data.

Cell-type heterogeneity (CTH) is a well-known major confounder in Epigenome-Wide Association Studies (EWAS), as these studies are generally performed in complex heterogeneous tissues such as blood, saliva or buccal swabs. Adjustment for CTH is critical and many statistical algorithms that adjust for this confounder have been developed and shown to significantly improve statistical inference. Among these, reference-based methods have been shown to significantly outperform reference-free ones. That reference-based methods do better can be attributed to them using solid prior information which takes the form of a DNA methylation reference matrix, consisting of representative DNAm profiles for the main cell-types in the tissue. Whilst generating such DNAm reference matrices can be challenging for many solid tissue-types, their construction can be relatively easily accomplished for tissues like blood, cord blood or saliva. For other solid tissues, computational strategies have enabled reasonable proxies for these DNAm reference matrices to be constructed, even at relatively high cellular resolution. More recently, using cell-sorting, Salas et al constructed a high resolution DNAm reference matrix for blood, consisting of 12 cell subtypes, thus increasing the resolution from the previous 7 common blood cell subtypes.

Importantly, recent studies have highlighted the need to perform adjustment for CTH at high cellular resolution. For instance, a component of Horvath's epigenetic clock has been shown to track an immunosenescence signature in which the ratio of naïve to mature T-cell fraction in blood decreases with age. As another example, using a 9 cell-type DNAm reference matrix for the olfactory epithelium, it has been shown that olfactory neuroblastomas consist of two subtypes, one originating from basal cells and another from immature neurons. However, the overall impact of performing CTH adjustment at a higher cellular resolution in common EWAS performed in blood is still underexplored.

Here we advance our understanding of the effect of cellular resolution on CTH adjustment, focusing mainly on blood EWAS. We use the resource from Salas et al to build a novel 12 blood cell-subtype DNAm reference matrix using an improved procedure that exclusively uses unmethylated cell-type specific markers. We validate this 12 cell-type DNAm reference matrix for blood using not only matched flow-cytometric data, but also in the context of real DNAm datasets where it correctly retrieves a well-known aging-associated immunosenescence signature. We then explore the impact of cellular resolution in CTH adjustment within the context of epigenetic clocks and biological aging, demonstrating that increased cellular resolution dampens the associations of biological age with factors such as gender and smoking. Finally, we show how increased cellular resolution is critical for discovering novel shifts in cell-type abundance with disease risk factors such as smoking.

### SUMMARY

In accordance with the purposes and benefits sets forth herein, a new and improved method is provided for determining percentages of twelve blood cell subtypes in a blood specimen. That method comprises, consists essentially of or consists of the steps of: (a) obtaining genomic DNA from the blood specimen of a human subject, (b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil, (c) comparing the unmethylated CG loci observed in (b) to unmethylated CG loci observed in genomic DNA collected from a reference group of human individuals and (d) correlating the unmethylated CG loci observed in (b) with the unmethylated CG loci observed in the reference group of human individuals to determine the percentage of twelve blood cell subtypes in the blood specimen. The CG loci are: cg21782213, cg10168351, cg03156020, cg17537714, cg13745692, cg04200580, cg15667493, cg21936454, cg15163862, cg19792189, cg10161198, cg19111287, cg05676874, cg03132824, cg17518643, cg09119854, cg11778706, cg04887735, cg22324641, cg08842268, cg09438865, cg08486432, cg04625250, cg08452021, cg20213624, cg02690807, cg22344918, cg18578898, cg19311567, cg16375820, cg08662013, cg11523325, cg21092682, cg23990235, cg24160371, cg01778017, cg05931551, cg03888645, cg21664107, cg06338239, cg01999608, cg08483099, cg23705316, cg11037382, cg11840035, cg21747310, cg20290061, cg07728865, cg19084822, cg03340102, cg03370360, cg04763150, cg11892665, cg21081729, cg24796422, cg05847670, cg00597139, cg12087639, cg24791846, cg01453386, cg14924828, cg17890852, cg07896398, cg00351957, cg25340205, cg02329886, cg21542081, cg06658453, cg08765214, cg11294011, cg03227316, cg23639055, cg25422758, cg04500050, cg15116227, cg09629758, cg02863748, cg03759549, cg25269402, cg08446606, cg19188904, cg09622285, cg26673070, cg04988216, cg21585800, cg09940675, cg14257017, cg15192986, cg14940779, cg17581877, cg11985063, cg05690666, cg20412398, cg19867897, cg23033014, cg03445419, cg17301153, cg04293820, cg14030593, cg26676468, cg03984633, cg25437005, cg19811231, cg26304744, cg27572854, cg22619531, cg09031281, cg00783080, cg00949794, cg09766370, cg27359629, cg05401393, cg08012294, cg04062032, cg11887733, cg03252427, cg12497611, cg06527889, cg13641221, cg12733242, cg18409707, cg02038353, cg12047375, cg03565732, cg18001026, cg23777479, cg15149205, cg22936341, cg07627628, cg04383721, cg05596912, cg02501773, cg15569739, cg24379742, cg00944599, cg15001636, cg13680246, cg12761472, cg19436514, cg00843117, cg17964575, cg26950756, cg27025274, cg16461337, cg13539891, cg23853980, cg07880190, cg26129027, cg15683542, cg10583651, cg05785919, cg22784669, cg18811564, cg03370491, cg18395354, cg11963313, cg26960322, cg10287321, cg27195778, cg04987479, cg02198280, cg09488807, cg07084675, cg00601124, cg27168830, cg25788647, cg02011162, cg22206842, cg20913547, cg24800373, cg15870556, cg04923194, cg14733637, cg17514643, cg03517570, cg07008893, cg14495399, cg03578212, cg26058722, cg23063536, cg19132501, cg03653880, cg24128943, cg10751207, cg10164437, cg12270000, cg07610771, cg27546582, cg00322928, cg26510178, cg21581865, cg05022030, cg22574467, cg01389234, cg26687322, cg19606289, cg06264696, cg20976977, cg14511575, cg05294459, cg25099065, cg14668581, cg10478600, cg15683107, cg07076850, cg15817752, cg07526731, cg05484949, cg13429555, cg26676273, cg06016162, cg02331158, cg16054240, cg26264767, cg08437936, cg07094228, cg27180005, cg07373578, cg13918228, cg02566312, cg09681537, cg17634650, cg25605180, cg00476430, cg18358155, cg01841368, cg04496578, cg02522485, cg16495530, cg07390370, cg25904998, cg05810879, cg24367295, cg19776833, cg14095525, cg16289232, cg05816398, cg02641941, cg26330304, cg04429341, cg11518040, cg03748473, cg04982372, cg27436324, cg06681449, cg25735823, cg06738507, cg10809134, cg11812782, cg25271915, cg05501327, cg22572158, cg04003505, cg22438885, cg12882261, cg23516026, cg23148481, cg25844471, cg10419892, cg15463664, cg08445080, cg15921515, cg24306984, cg23539484, cg03922997, cg05487307, cg27340845, cg03217619, cg13477627, cg15033776, cg15164173, cg08358686, cg08888548, cg00678912, cg15560912, cg07003611, cg16255804, cg07833643, cg26451108, cg08337454, cg15908108, cg17642929, cg03833097, cg18857045, cg23411838, cg00409021, cg17290867, cg16044562, cg02105817, cg18459351, cg05106785, cg10208609, cg16901161, cg02033323, cg24998002, cg07461115, cg04311057, cg06026449, cg00052246, cg02148547, cg02203103, cg20063728, cg20813776, cg13475329, cg13211380, cg20652042, cg10651616, cg02826841, cg05892024, cg25918166, cg03267512, cg27298253, cg18327204, cg19653890, cg02319946, cg18073938, cg21023071, cg07661702, cg16613029, cg13452923, cg11749308, cg10220401, cg08657911, cg17523148, cg24898812, cg04017239, cg22250168, cg11249120, cg04467549, cg01850245, cg11693925, cg04705866, cg24240349, cg01923140, cg12695059, cg05036909, cg05982017, cg26429965, cg09856271, cg08243981, cg14860676, cg22755579, cg05689028, cg15661018, cg05471842, cg00639010, cg19253785, cg16438469, cg03501128, cg04657000, cg15213052, cg24422171, cg19850895, cg10587583, cg05478809, cg25448766, cg22556056, cg03077364, cg18329839, cg07702659, cg17351376, cg13487687, cg07707056, cg20697519, cg04394724, cg12742224, cg00644351, cg13693582, cg23172664, cg16596052, cg14027234, cg01002223, cg04928784, cg16253157, cg12043508, cg16704246, cg04047276, cg16070350, cg20330224, cg02053685, cg00790439, cg26796793, cg20492070, cg12152193, cg16780406, cg06419846, cg18844901, cg19792099, cg14110920, cg21631387, cg20560862, cg17445453, cg02865554, cg07756788, cg09799856, cg17829179, cg25235205, cg11020586, cg20587232, cg04249416, cg19764973, cg24818060, cg06421734, cg11649969, cg04148640, cg01355027, cg15301710, cg01511347, cg17960717, cg00006459, cg15873326, cg11668148, cg09596645, cg08538069, cg02233915, cg22106847, cg07033790, cg05840984, cg10220104, cg17030380, cg02803925, cg14084609, cg16909341, cg05736642, cg13531735, cg01404879, cg21503771, cg15662508, cg05906696, cg09803743, cg12910830, cg15090899, cg22034357, cg17105176, cg24732344, cg15997319, cg09010228, cg11262246, cg05089849, cg22954836, cg02179849, cg25293601, cg24057263, cg01048814, cg08077807, cg00637085, cg17542165, cg13123707, cg26156120, cg18852698, cg18629096, cg27274718, cg05875239, cg13617280, cg24410690, cg05355684, cg18584272, cg18546155, cg26275360, cg14563201, cg08326410, cg02855870, cg15956469, cg27040968, cg10206039, cg09932794, cg09304968, cg00949861, cg02847139, cg25386954, cg04737115, cg03368758, cg14058851, cg23369683, cg14047092, cg27495654, cg22829007, cg16995933, cg01416168, cg08530578, cg07543798, cg12851731, cg15646529, cg03538296, cg13257250, cg17211761, cg18948661, cg13598219, cg22668643, cg09134152, cg21180201, cg08907176, cg03559700, cg21275838, cg05508074, cg18857340, cg12973514, cg16360310, cg24137511, cg16316183, cg25600606, cg18646784, cg05956380, cg16683571, cg18330536, cg17781418, cg13468144, cg26849760, cg23338668, cg13155300, cg24392753, cg22400420, cg10934870, cg14095610, cg01923724, cg06500418, cg04179951, cg16597887, cg04036920, cg15715903, cg01701207, cg08218512, cg07619820, cg01699630, cg25790531, cg19619956, cg26396370, cg05170732, cg08091050, cg09622624, cg02379348, cg25576801, cg27393431, cg09601944, cg24131359, cg26979142, cg04682832, cg25757820, cg26789411, cg17331920, cg00434123, cg16107565, cg23911433, cg12578084, cg15810023, cg08771884, cg07993518, cg20030158, cg20077668, cg27646850, cg15173892, cg07617198, cg10480329, cg20374292, cg00771778, cg18066690, cg02647842, cg20290017, cg02244028, cg06636137, cg25605773, cg22675702, cg08844422, cg26872907, cg23244761, cg23373670, cg14172797, cg26201751, cg02290460, cg18356337, cg00908794, cg08965078, cg16636767, cg09592164, cg17195809, cg19336573, cg03135975, cg05065075, cg01407558, cg08097676, cg23312369, cg11620863, cg10624395, cg03556458, cg14116399, cg19501306, cg02007480, cg23950707, cg14458725, cg14898005, cg01950767, cg18844042, cg05923857, cg01842756, cg03963853, cg11183587, cg21185441, cg02647748, cg14806960, and cg04851465.

In one or more of the many possible embodiments of the method, the method may further include amplifying the genomic DNA of the blood specimen by a polymerase chain reaction.

In one or more of the many possible embodiments, the method may further include using a regression algorithm to correlate the unmethylated CG loci observed in (b) with the unmethylated GC loci observed in the reference group of human individuals.

In one or more of the many possible embodiments, the observing of the unmethylated CG loci, in the genomic DNA of the human subject includes hybridizing the genomic DNA to a beadchip to create a genomic DNA hybridized bead chip and then imaging the genomic DNA hybridized beadchip.

In accordance with yet another aspect, the new and improved method comprises, consists essentially of or consists of the steps of: (a) obtaining genomic DNA from the blood specimen of a human subject, (b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil, (c) comparing the unmethylated CG loci observed in (b) to unmethylated CG loci observed in genomic DNA collected from a reference group of human individuals and (d) correlating the unmethylated CG loci observed in (b) with the unmethylated CG loci observed in the reference group of human individuals to determine the percentage of the twelve blood cell subtypes in the blood specimen wherein the CG loci are: cg13745692, cg15667493, cg21936454, cg15163862, cg19111287, cg03132824, cg09119854, cg04887735, cg08486432, cg18578898, cg16375820, cg11523325, cg24160371, cg05931551, cg03888645, cg11840035, cg21747310, cg07728865, cg19084822, cg17894779, cg24503957, cg14138047, cg00694660, cg13484324, cg01364581, cg02725065, cg07133479, cg02280893, cg03069943, cg07643762, cg21200667, cg06828418, cg17824906, cg00378228, cg24463664, cg18984002, cg10091155, cg09177577, cg11840104, cg15958839, cg16192197, cg13104185, cg15009294, cg00629382, cg00705661, cg00208274, cg24211304, cg20558320, cg04073934, cg05158109, cg21081729, cg12087639, cg24791846, cg02329886, cg11294011, cg23639055, cg25422758, cg09622285, cg26673070, cg04988216, cg09940675, cg15192986, cg23033014, cg14030593, cg26676468, cg19699264, cg17170133, cg12037509, cg03380342, cg24736010, cg17436460, cg18469148, cg24496563, cg21039188, cg05506809, cg00712106, cg12646067, cg03184452, cg07862744, cg14633252, cg17282428, cg02587120, cg00551398, cg26009797, cg00086283, cg09275783, cg15106420, cg22197708, cg15003763, cg01782059, cg13894852, cg09131332, cg07188551, cg26252958, cg06706029, cg13394713, cg26603598, cg05201777, cg11809342, cg23743708, cg25437005, cg19811231, cg26304744, cg27572854, cg27359629, cg08012294, cg11887733, cg12047375, cg23777479, cg15149205, cg07627628, cg24379742, cg00944599, cg15001636, cg13680246, cg12761472, cg00843117, cg17964575, cg26950756, cg07880190, cg26129027, cg15683542, cg10583651, cg18289508, cg23005797, cg07784293, cg09222732, cg16497398, cg02619107, cg12419766, cg07340499, cg06828613, cg23635663, cg23506728, cg27297043, cg24525744, cg06288253, cg25404597, cg21814995, cg00292305, cg19622138, cg18303608, cg27518047, cg18835815, cg18051361, cg10295609, cg05519606, cg23271530, cg13011976, cg09449150, cg18395354, cg26960322, cg10287321, cg00601124, cg04923194, cg14733637, cg14495399, cg03653880, cg24128943, cg26510178, cg20976977, cg07316730, cg00148025, cg25382573, cg15829423, cg08220696, cg26383838, cg22084515, cg07960360, cg09241714, cg08761324, cg01884715, cg07044414, cg03680932, cg06369076, cg04330057, cg00083596, cg27576271, cg13678009, cg14101687, cg20561261, cg27157619, cg23900696, cg12208691, cg18822703, cg04329659, cg13551330, cg03822267, cg27416848, cg00087511, cg19277389, cg03717570, cg06099459, cg06541733, cg23731501, cg02079700, cg06419212, cg18861762, cg27317694, cg06013895, cg25099065, cg07076850, cg05484949, cg13429555, cg07373578, cg13918228, cg17634650, cg00476430, cg02522485, cg19776833, cg05816398, cg02641941, cg26330304, cg27436324, cg25735823, cg10809134, cg27184438, cg10361659, cg03653817, cg03328673, cg17453767, cg08246318, cg00649632, cg13276502, cg20847766, cg03386536, cg01598421, cg14422093, cg16098726, cg03238899, cg08986727, cg00374717, cg00863309, cg10678215, cg14905514, cg15552491, cg04074908, cg25107522, cg07433769, cg16586807, cg02563364, cg11972721, cg12796332, cg22528123, cg16417374, cg23325384, cg06916059, cg09299082, cg05776861, cg01502811, cg05501327, cg22572158, cg23516026, cg25844471, cg08445080, cg15921515, cg03922997, cg15033776, cg07003611, cg16255804, cg03833097, cg05106785, cg10208609, cg02033323, cg00052246, cg02148547, cg26408382, cg08562111, cg22360028, cg09490124, cg09197075, cg18124517, cg05878535, cg23519969, cg17498667, cg00463840, cg09642378, cg02397552, cg19876232, cg12485044, cg13345122, cg17392230, cg01554304, cg25557227, cg23541811, cg03873220, cg16685586, cg02979010, cg06840801, cg01865118, cg01436550, cg11812071, cg16637690, cg22980427, cg22577252, cg07805771, cg24599709, cg02272851, cg24847035, cg07793163, cg20063728, cg20652042, cg05892024, cg25918166, cg16613029, cg13452923, cg11249120, cg04467549, cg01850245, cg04705866, cg24240349, cg12695059, cg05982017, cg09856271, cg22755579, cg05689028, cg05471842, cg00639010, cg03501128, cg04657000, cg24037746, cg00460916, cg24618739, cg09736286, cg12983578, cg20417024, cg23320693, cg13805183, cg08035195, cg09096234, cg11161150, cg17542594, cg10335493, cg14776168, cg02763540, cg16946840, cg03315940, cg01356867, cg02586198, cg25503323, cg12524168, cg26509305, cg07109551, cg02205013, cg19884600, cg09088625, cg03534847, cg08877257, cg12159213, cg05905180, cg24422171, cg10587583, cg05478809, cg22556056, cg07702659, cg17351376, cg07707056, cg20697519, cg04394724, cg00644351, cg13693582, cg23172664, cg16596052, cg14027234, cg01002223, cg16253157, cg12043508, cg16704246, cg12152193, cg06419846, cg19792099, cg14110920, cg20560862, cg07756788, cg25235205, cg01949403, cg14507427, cg05496363, cg09872088, cg23463186, cg14468508, cg20367218, cg12221970, cg26856443, cg09628822, cg12655059, cg08585614, cg26668000, cg16359142, cg11067179, cg09127592, cg10365743, cg19556572, cg09586583, cg22213242, cg03788408, cg06748978, cg26354221, cg14537031, cg20681184, cg19764973, cg17960717, cg00006459, cg11668148, cg09596645, cg22106847, cg07033790, cg02803925, cg14084609, cg16909341, cg05736642, cg13531735, cg12910830, cg15090899, cg24732344, cg08077807, cg00637085, cg26156120, cg21237481, cg23153546, cg25203627, cg18550847, cg04538585, cg06680065, cg00391067, cg11197258, cg16362140, cg06315149, cg00170714, cg07410597, cg08061334, cg14209186, cg25578728, cg14697334, cg27000590, cg25381747, cg24706564, cg20315954, cg07766916, cg17732216, cg19788934, cg08969102, cg15427040, cg17738684, cg19968199, cg24459209, cg20240243, cg16472853, cg07201450, cg01820584, cg27274718, cg05875239, cg13617280, cg24410690, cg05355684, cg26275360, cg08326410, cg15956469, cg27040968, cg10206039, cg09304968, cg25386954, cg03368758, cg14058851, cg01416168, cg03538296, cg17211761, cg21180201, cg21275838, cg16360310, cg24137511, cg16316183, cg04992852, cg25846372, cg07802362, cg13254203, cg01112784, cg05253716, cg15705930, cg18087143, cg23001370, cg07873128, cg10830795, cg03891962, cg09278623, cg03055021, cg25029657, cg17169982, cg10157558, cg00515905, cg23617121, cg27309871, cg21777154, cg07123481, cg22917487, cg16220743, cg03956353, cg04718702, cg01830256, cg11790417, cg25600606, cg13468144, cg23338668, cg22400420, cg10934870, cg01923724, cg04036920, cg01699630, cg25790531, cg19619956, cg26396370, cg08091050, cg24131359, cg25757820, cg17331920, cg23911433, cg09694051, cg13633625, cg17078883, cg17322118, cg07079231, cg01632240, cg13785123, cg02969426, cg17567562, cg15457217, cg15169266, cg07052231, cg16102040, cg11525409, cg13618969, cg00591421, cg09166556, cg06494464, cg07804973, cg25693317, cg13589463, cg21772826, cg11809958, cg02279108, cg06784232, cg05492904, cg14523804, cg14249174, cg05006942, cg18619616, cg25539505, cg09948350, cg27466532, cg17547928, cg10480329, cg18066690, cg02244028, cg22675702, cg26872907, cg23244761, cg16636767, cg10624395, cg05923857, cg03963853, cg04851465, cg21459713, cg05242065, cg08908131, cg27366072, cg19301114, cg24788483, cg07002540, cg26124526, cg01041239, cg18589858, cg07125725, cg12655112, cg16356013, cg25376491, cg25898577, cg20641531, cg14043316, cg05452742, cg16728323, cg08694574, cg23719713, cg00646216, cg13290254, cg25517015, cg16766036, cg20918393, cg25291653, cg07505901, cg03567939, cg25765315, cg14872074, cg18770149, cg23391288, cg21983607, cg12037947, cg26953469, cg01024962, cg02780988, and cg14428166.

In one or more of the many possible embodiments of the method, the method may further include amplifying the genomic DNA of the blood specimen by a polymerase chain reaction.

In one or more of the many possible embodiments, the method may further include using a regression algorithm to correlate the unmethylated CG loci observed in (b) with the unmethylated GC loci observed in the reference group of human individuals.

In one or more of the many possible embodiments, the observing of the unmethylated CG loci, in the genomic DNA of the human subject includes hybridizing the genomic DNA to a beadchip to create a genomic DNA hybridized bead chip and then imaging the genomic DNA hybridized beadchip.

In accordance with yet another aspect, the new and improved method comprises, consists essentially of or consists of the steps of: (a) obtaining genomic DNA from the blood specimen of a human subject, (b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil, (c) comparing the unmethylated CG loci observed in (b) to unmethylated CG loci observed in genomic DNA collected from a reference group of human individuals and (d) correlating the unmethylated CG loci observed in (b) with the unmethylated CG loci observed in the reference group of human individuals to determine the percentage of the blood cell subtypes in the blood specimen wherein the CG loci are: cg13745692, cg15667493, cg21936454, cg15163862, cg19111287, cg03132824, cg09119854, cg04887735, cg08486432, cg18578898, cg16375820, cg11523325, cg24160371, cg05931551, cg03888645, cg11840035, cg21747310, cg07728865, cg19084822, cg21081729, cg12087639, cg24791846, cg02329886, cg11294011, cg23639055, cg25422758, cg09622285, cg26673070, cg04988216, cg09940675, cg15192986, cg23033014, cg14030593, cg26676468, cg25437005, cg19811231, cg26304744, cg27572854, cg27359629, cg08012294, cg11887733, cg12047375, cg23777479, cg15149205, cg07627628, cg24379742, cg00944599, cg15001636, cg13680246, cg12761472, cg00843117, cg17964575, cg26950756, cg07880190, cg26129027, cg15683542, cg10583651, cg18395354, cg26960322, cg10287321, cg00601124, cg04923194, cg14733637, cg14495399, cg03653880, cg24128943, cg26510178, cg20976977, cg25099065, cg07076850, cg05484949, cg13429555, cg07373578, cg13918228, cg17634650, cg00476430, cg02522485, cg19776833, cg05816398, cg02641941, cg26330304, cg27436324, cg25735823, cg10809134, cg05501327, cg22572158, cg23516026, cg25844471, cg08445080, cg15921515, cg03922997, cg15033776, cg07003611, cg16255804, cg03833097, cg05106785, cg10208609, cg02033323, cg00052246, cg02148547, cg20063728, cg20652042, cg05892024, cg25918166, cg16613029, cg13452923, cg11249120, cg04467549, cg01850245, cg04705866, cg24240349, cg12695059, cg05982017, cg09856271, cg22755579, cg05689028, cg05471842, cg00639010, cg03501128, cg04657000, cg24422171, cg10587583, cg05478809, cg22556056, cg07702659, cg17351376, cg07707056, cg20697519, cg04394724, cg00644351, cg13693582, cg23172664, cg16596052, cg14027234, cg01002223, cg16253157, cg12043508, cg16704246, cg12152193, cg06419846, cg19792099, cg14110920, cg20560862, cg07756788, cg25235205, cg19764973, cg17960717, cg00006459, cg11668148, cg09596645, cg22106847, cg07033790, cg02803925, cg14084609, cg16909341, cg05736642, cg13531735, cg12910830, cg15090899, cg24732344, cg08077807, cg00637085, cg26156120, cg27274718, cg05875239, cg13617280, cg24410690, cg05355684, cg26275360, cg08326410, cg15956469, cg27040968, cg10206039, cg09304968, cg25386954, cg03368758, cg14058851, cg01416168, cg03538296, cg17211761, cg21180201, cg21275838, cg16360310, cg24137511, cg16316183, cg25600606, cg13468144, cg23338668, cg22400420, cg10934870, cg01923724, cg04036920, cg01699630, cg25790531, cg19619956, cg26396370, cg08091050, cg24131359, cg25757820, cg17331920, cg23911433, cg10480329, cg18066690, cg02244028, cg22675702, cg26872907, cg23244761, cg16636767, cg10624395, cg05923857, cg03963853, and cg04851465.

In one or more of the many possible embodiments of the method, the method may further include amplifying the genomic DNA of the blood specimen by a polymerase chain reaction.

In one or more of the many possible embodiments, the method may further include using a regression algorithm to correlate the unmethylated CG loci observed in (b) with the unmethylated GC loci observed in the reference group of human individuals.

In one or more of the many possible embodiments, the observing of the unmethylated CG loci, in the genomic DNA of the human subject includes hybridizing the genomic DNA to a beadchip to create a genomic DNA hybridized bead chip and then imaging the genomic DNA hybridized beadchip.

In the following description, there are shown and described several preferred embodiments of the method of determining percentages of blood cell subtypes in a blood specimen. As it should be realized, that method is capable of other, different embodiments and its several details are capable of modification in various, obvious aspects all without departing from the method as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate certain aspects of the method and together with the description serve to explain certain principles thereof.

FIG. 1A illustrates construction and validation of the 12 blood cell-type hypoDNAm reference matrix. Left panel: Example of a CpG's DNAm profile in the DNAm reference matrix, marking T-regulatory (Treg) cells. The y-axis labels the cell-types, x-axis labels the DNAm value, and the number of samples of each cell-type (i.e. in each boxplot) is shown on the y-axis. Right panel: The DNAm reference matrix for 12 blood cell subtypes encompassing 600 CpGs (i.e. 50 markers per cell-type).

FIG. 1B is ten scatterplots of true fractions vs estimated fractions for 10 blood cell subtypes using the EPIC DNAm data from 10 artificial mixtures where the underlying mixing proportions were known. For each estimated cell-type we display the R-value (Pearson Correlation Coefficient) and root mean square error (RMSE).

FIG. 1C is as in FIG. 1B, but now for 6 whole blood EPIC DNAm profiles with matched FACS cell-counts for 7 cell-types, as shown. Here f(True) denotes the cell-fraction from FACS.

FIG. 1D is as in FIG. 1C, but now for 144 peripheral blood EPIC DNAm profiles with matched FACS counts for 3 cell-types.

FIG. 2A is ten scatterplots of true fractions vs estimated fractions for ten blood cell subtypes using the EPIC DNAm data from ten artificial mixtures where the underlying mixtures were known. The cell type fractions were estimated using the twelve blood cell-type reference DNAm matrix restricted to 450k probes. For each estimated cell-type we display the R-value (Pearson Correlation Coefficient) and root mean square error (RMSE).

FIG. 2B is seven scatterplots as in FIG. 1A but now for six whole blood EPIC DNAm profiles with matched FACS cell-counts for seven cell-types, as shown. Here f(True) denotes the cell-fraction from FACS.

FIG. 3A is a heatmap of associations between blood cell-type fractions and age in each of 17 cohorts. Right panels illustrate an example, displaying the change in CD4+ and CD8+ naïve T-cell fractions with age. Number of data points in each boxplot/age-bin is shown below age-bin. Dashed line and P-value is from a linear regression.

FIG. 3B is a heatmap of associations between blood cell-type fractions and sex in each of 14 cohorts with gender information.

FIG. 3C is a Heatmap of associations between blood cell-type fractions and smoking in each of 9 cohorts with smoking-status information.

FIG. 4A are barplots displaying for each of the 21 datasets, the proportion of samples falling in one of five age-bins, as shown. The total number of samples in each study is displayed at the top of the bars.

FIG. 4B is boxplots displaying the full range of ages in each of the twenty cohorts.

FIG. 5 is three scatterplots of eosinophil fraction (eosF, y-axis) vs age (x-axis) for blood samples from childhood cancer survivors, stratified by type of treatment: left panel displays patients who only received radiotherapy (RT), middle panel displays patients that only received chemotherapy (CT), right panel displays patients who received both RT and CT. The number of samples in each strata is given above plot. Within the plots we provided the change in eosinophil fraction across a 40-year age gap, as well as the associated P-value from a linear regression.

FIG. 6 is barplots displaying for each of the 21 datasets, the proportion of males and females. The total number of samples in each study is displayed at the top of the bars.

FIG. 7A is barplots displaying each of 10 datasets with smoking information available, the smoking status of the subjects at sample draw. The total number of samples in each study is displayed at the top of the bars.

FIG. 7B is violin plots depicting the distribution of Body Mass Index (BMI) values in the five cohorts where BMI was available. The number of samples in each cohort is given.

FIG. 8 is a heatmap displaying the statistical significance and directionality of associations of blood cell-type fractions with BMI for five different cohorts. The number of samples in each corhort is given at the top of the heatmap.

FIG. 9 is a boxplots of cell-type fractions (CTFs, y-axis) against Covid-19 disease severity (x-axis) for all those blood cell subsets for which an association was statistically significant. The number of samples in each disease group is given below x-axis. P-value is two-tailed and from a linear regression of CTF against disease group labeled as 0 (control/negative), 1 (mild) and 2 (severe).

FIG. 10 is DNAmAge plotted vs chronological age for each of nineteen datasets. The P-value and R² from a linear regression is given.

FIG. 11 is DNAmAge plotted vs chronological age for each of 18 datasets. The P-value and R² from a linear regression is given.

FIG. 12 is is DNAmAge plotted vs chronological age for each of 17 datasets. The P-value and R² from a linear regression is given.

FIGS. 13A-13C are analysis of cell-type heterogeneity correlation to biological age acceleration measurements such as Extrinsic epigenetic age (EAA) and Instinct epigenetic age adjusted by cell type proportions estimates through 7 and 12 cell deconvolution methods. 13A) Barplots of t-statistics (x-axis) of association of 3 separate measures of Horvath's clock age-acceleration (Extrinsic Age Acceleration—EAA, Intrinsic Age Acceleration with adjustment for 7 immune-cell subtypes—IAA7 and adjustment for 12 immune-cell subtypes—IAA12) with sex. Cohorts are labeled on the y-axis. Paired one-tailed Wilcoxon rank sum test P-values between the 3 measures are given to assess if adjustment for 7 and 12 immune cell subtypes decreases the t-statistics. 13B) Boxplots of PhenoAge's EAA, IAA7 and IAA12 against Covid-19 disease severity in the Barturen et al dataset. The number of samples in each disease group is indicated below x-axis. t-statistic and P-value shown are from a linear regression encoding disease state as 0 (negative/control), 1 (mild disease) and 2 (severe disease). 13C) As 13B, but against obesity (BMI) stratified as normal (N, BMI<25), mild (25<BMI<30), medium (30<BMI<35) and extreme (BMI>35) in the TD10k cohort.

FIG. 14A is barplots of t-statistics (x-axis) of association of three separate measures of PhenoAge's clock age-acceleration (Extrinsic Age Acceleration-EAA, Intrinsic Age Acceleration with adjustment for seven immune-cell subtypes—IAA7 and adjustment for twelve immune-cell subtypes—IAA12) with sex. Cohorts are labeled on the y-axis. Paired one-tailed Wilcoxon rank sum test P-values between the three measures are given to assess if adjustment for seven and twelve immune cell subtypes decreases the t-statistics.

FIG. 14B is boxplots of Horvath clock's EAA, IAA7 and IAA12 against Covid-19 disease severity in the Barturen et al dataset. The number of samples in each disease group is indicated below the x-axis. T-statistic and P-value shown are from a linear regression encoding disease state as 0 (negative/control), 1 (mild disease) and 2 (severe disease).

FIG. 14C is as in FIG. 10B, but against obesity (BMI) stratified as normal (N, BMI<25), mild (25<BMI<30), medium (30<BMI<35) and extreme (BMI>35) in the TD10k cohort.

Reference will now be made in detail to the present preferred embodiments of the method.

### DETAILED DESCRIPTION

### Construction and Validation of a 12 Blood Cell Subtype DNAm Reference Matrix

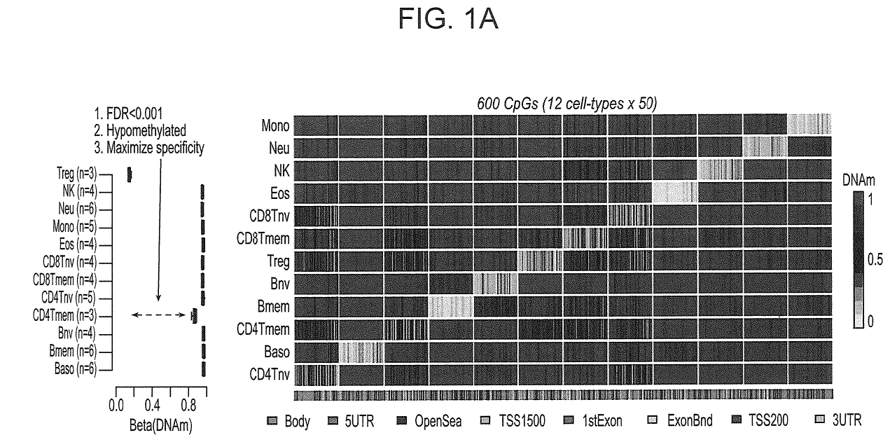

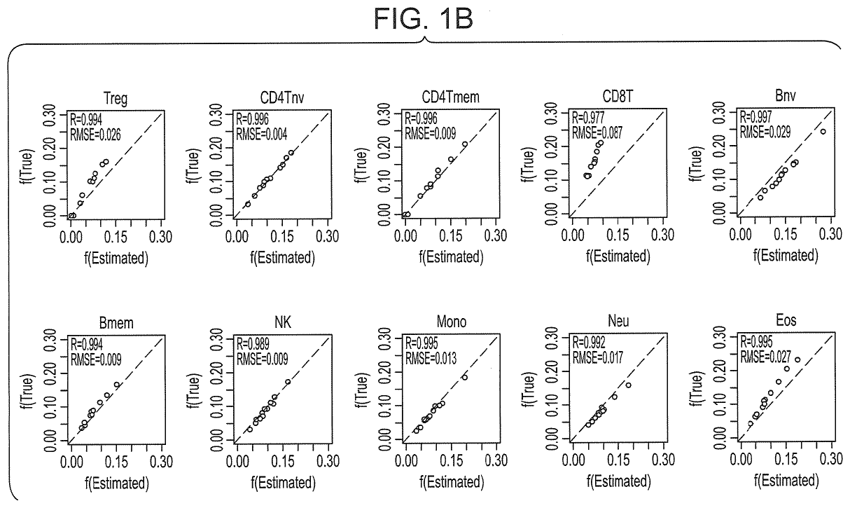

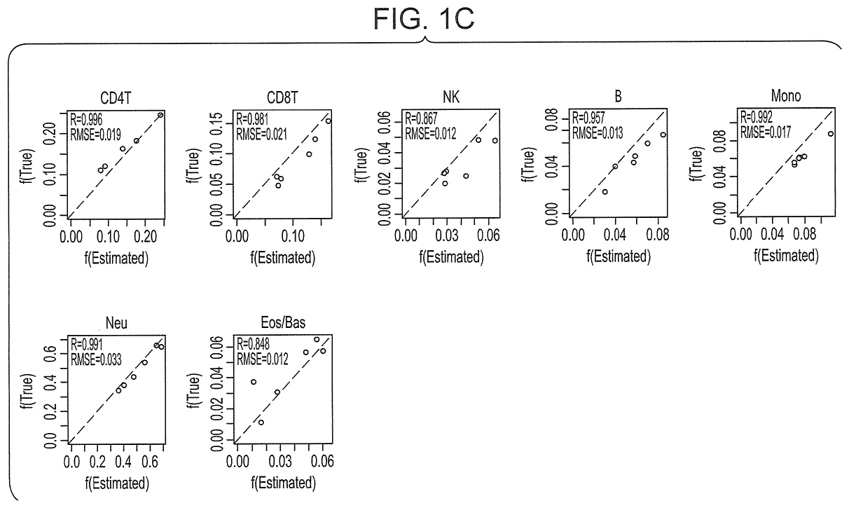

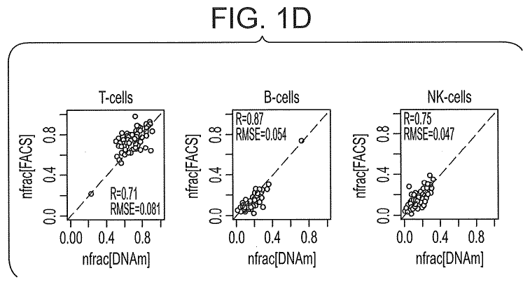

We constructed a novel DNAm reference matrix encompassing 12 blood cell subtypes (monocytes, neutrophils, eosinophils, basophils, naïve and memory CD4+ T-cells, naïve and memory CD8+ T-cells, naïve and memory B-cells, natural killer (NK) and T-regulatory (Tregs) cells) using the EPIC DNAm profiles of FACS-sorted cells from Salas et al. Since cell-type specific marker genes display a very strong preference for unmethylated promoters and enhancers in the corresponding cell-types, we decided to construct a DNAm reference matrix focusing on CpGs specifically unmethylated in each cell-type (Methods). For each of the 12 cell-types, we selected CpGs significantly hypomethylated (FDR<0.001) in the given cell-type relative to all the rest, subsequently ranking them according to the difference in average DNAm, so as to ensure maximum separability (See FIG. 1A).

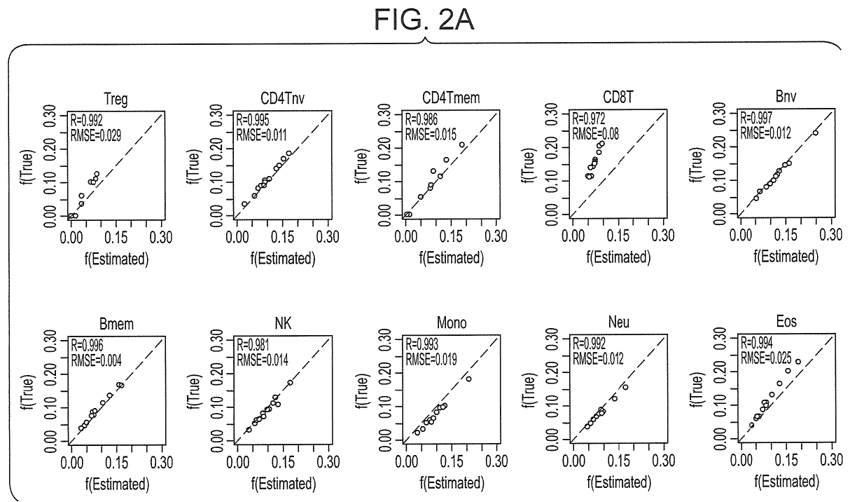

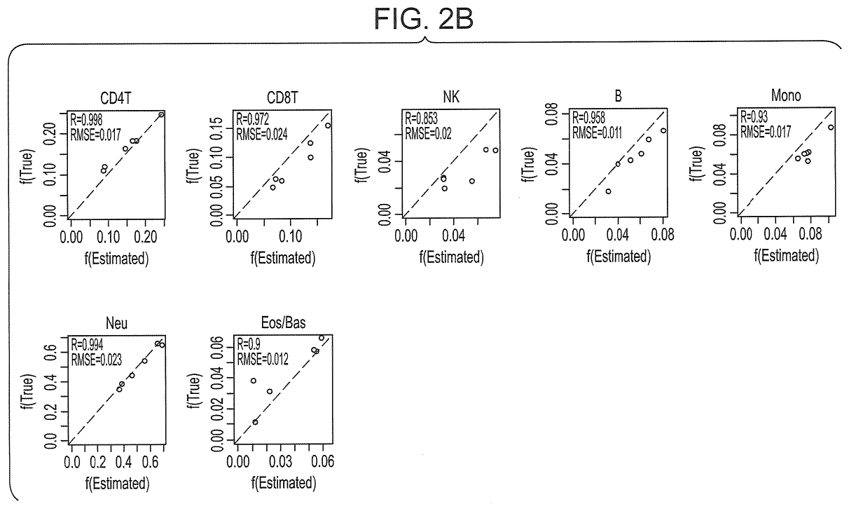

We verified that by selecting the top-ranked 50 CpGs for each cell-type (i.e. a total of 600 CpGs), that these displayed very significant hypomethylation and relatively big differences in average DNAm, as required (FIGS. 1A, 2A and 2B). About half of the 600 CpGs mapped to gene-bodies, whilst the other half mapped predominantly to inter-genic regions and shores/shelves upstream of the TSS (FIG. 1A).

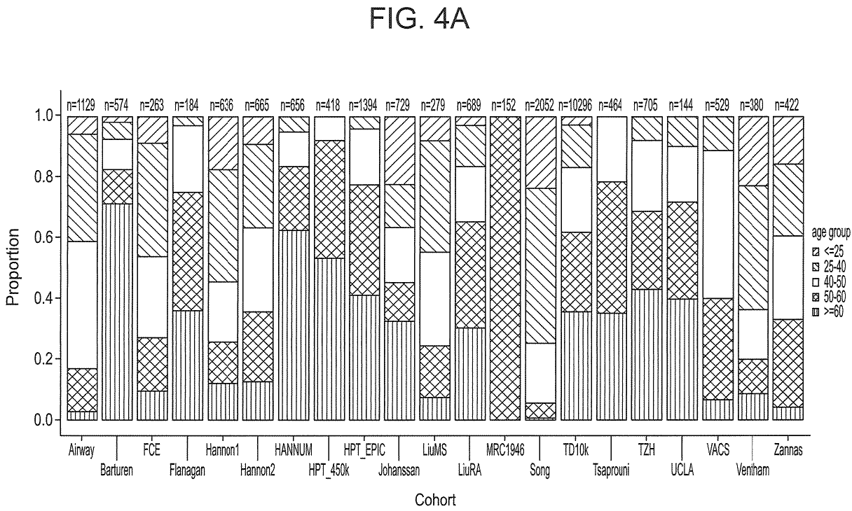

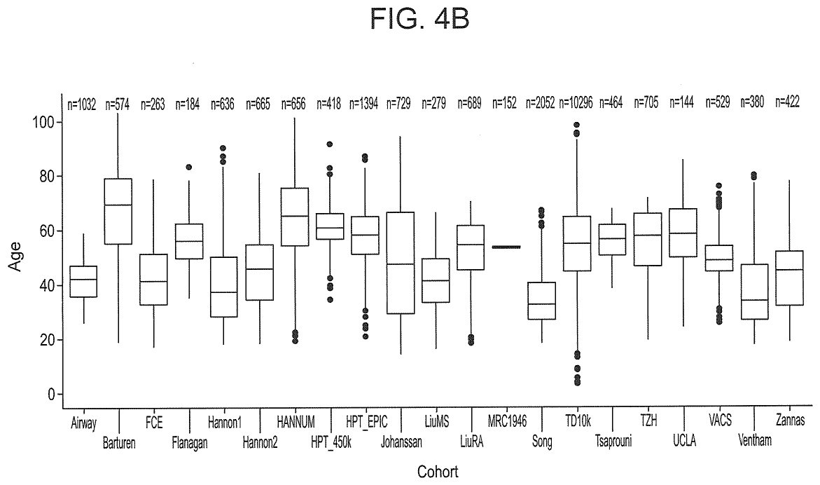

To validate the 12 blood cell-subtype DNAm reference matrix, we applied it in conjunction with the Robust Partial Correlation (RPC) framework implemented in EpiDISH to estimate cell-type fractions in 12 artificial mixtures where the proportions of 10 underlying blood cell-subtypes used in these mixtures are known. Pearson correlations and root mean square error (RMSE) were excellent for all tested cell-types, thus validating the DNAm reference matrix (FIG. 1B). To further validate it, we compared estimated cell-type fractions with matched flow-cytometric counts in two independent datasets, one encompassing 6 blood samples with matched counts for 7 blood cell-subtypes, and another encompassing 144 peripheral blood samples with matched counts for 3 types of lymphocytes (Methods). For both datasets, Pearson correlation R-values and RMSE were reasonably high, further attesting to the quality of our DNAm reference matrix (FIGS. 1C and 1D). For completeness, we repeated the construction of a 12 cell-type DNAm reference matrix, but this time restricting to Illumina 450k probes, resulting in a separate 600 CpG×12 cell-type DNAm reference matrix (FIGS. 4A and 4B). We successfully validated this DNAm reference matrix in artificial blood mixtures and in blood samples with matched flow-cytometric counts (FIGS. 2A and 2B).

### Validation Against an Age-associated Immunosenescence Signature

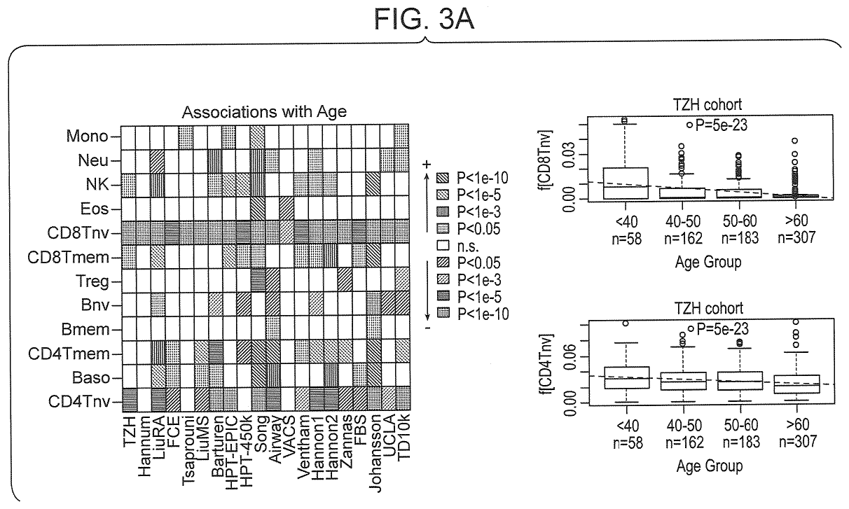

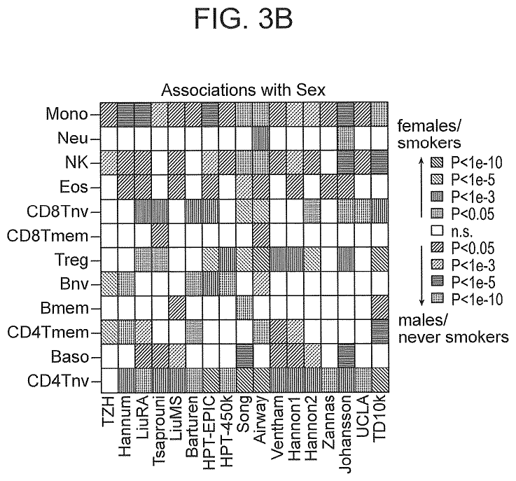

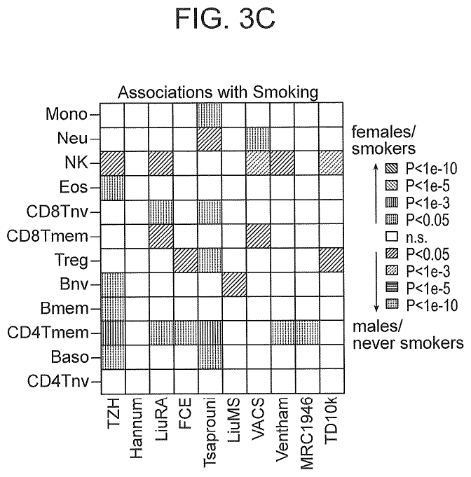

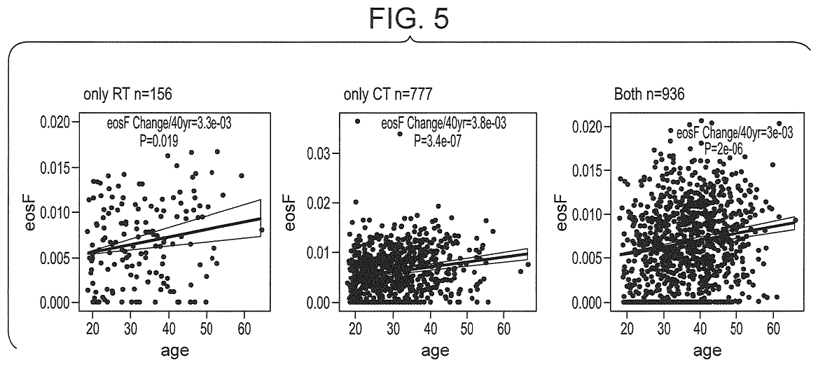

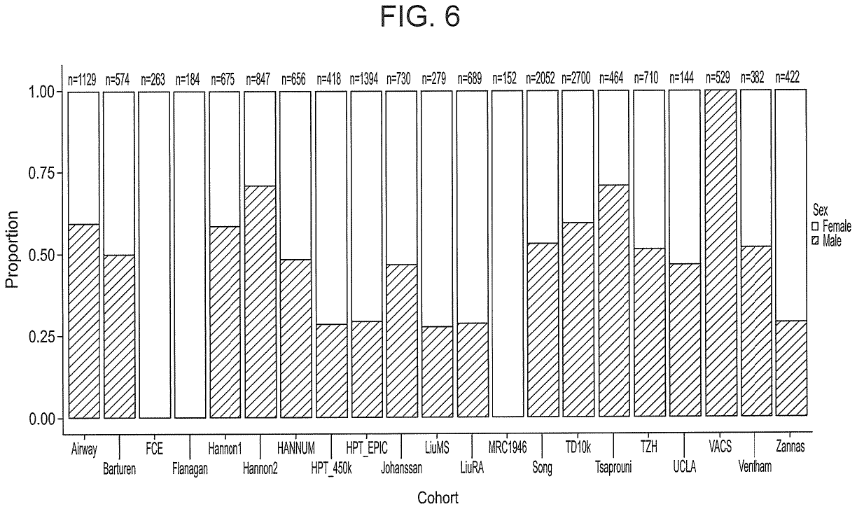

Because large DNAm datasets with matched flow-cytometric counts for as many as 12 blood cell-types are not available, it is paramount to seek other means to validate the high resolution DNAm reference matrix on real data. To this end, we asked if our DNAm reference matrix would be able to correctly capture a well-known age-associated immunosenescence signature characterized by a decreased naïve to mature T-cell fraction ratio. We note that an age-associated shift in abundance from naïve CD4+ and CD8+ T-cells to corresponding mature T-cell subtypes has been amply validated using single-cell RNA-sequencing data, and that it corresponds to a well-known age-associated signature of immunosenescence. We estimated fractions for all 12 blood cell subtypes in 21 independent whole/peripheral blood cohorts, encompassing 22,990 samples and two versions of the Illumina DNAm array (EPIC & 450k) (FIG. 5). For most studies, the chronological age distribution was wide, except for one study where all individuals were of the same age (FIGS. 4A, 4B and 5). Validating our DNAm reference matrix, we observed a strong consistent reduction in the naïve CD8+ T-cell population with age across all 20 studies. For CD4T+ cells, the reduction of the naïve subset was also evident in 16 out of 20 studies (FIG. 3A). Correspondingly, there was a trend for the memory T-cell subsets to increase with age (FIG. 3A). Thus, these data support the view that our 12 cell-type DNAm reference matrix is accurate in so far as it can capture subtle known shifts within immune T-cell subsets.

### Meta-analysis Reveals Novel Associations of Cell-type Fractions With Age

A meta-analysis over many datasets can also reveal potentially novel associations or strengthen previous observations. For instance, our meta-analysis revealed a clear trend for basophil and NK cell fractions to increase with age (FIG. 3A). We attribute variations between cohorts to the small effect sizes involved or to intrinsic differences between studies, thus highlighting the importance and power of performing meta-analyses. Indeed, that the overall NK fraction may increase with age is very plausible given reports that the differentiated mature subset displays age-associated increases. That basophils become more abundant with increased age is also supported by a study comparing basophil counts in bone marrow and spleens of old versus young mice.

Our meta-analysis also revealed that the eosinophil fraction did not change significantly with age, except for the studies of Song et al, which profiled DNAm in whole blood of childhood cancer survivors, and the Veteran Aging Cohort Study (VACS), where however the association was only very marginal (FIG. 3A). The meta-analysis result is consistent with a major eosinophil count study in over 11,000 subjects, which concluded that eosinophil counts do not change with age beyond the age of puberty. Indeed, all cohorts analyzed did not include samples pre-puberty and no samples less than 18 years of age (FIG. 4B). Thus, the fact that in Song et al we observed an increased eosinophil count with age suggests that chemotherapy may alter hematopoiesis in a manner that promotes eosinophilia with age.

### Meta-analysis Reveals Associations of Cell Counts with Sex

A total of 17 studies profiled blood in both males and females (SI FIG. S3). The meta-analysis revealed increased monocyte, eosinophil, basophil and NK-cell fractions in males, and increased T-regulatory and naïve T-cell subset fractions in females (FIG. 2b). The increased eosinophil fraction in males is consistent with an eosinophil count study in over 11,000 Austrian subjects which derived an odds ratio (OR) of male vs female eosinophil counts of 1.36. The increased NK-cell fraction in males is also supported by a study that used single-cell RNA-sequencing to compare immune-cell fractions between males and females. This same study also observed a marginal increase of the monocyte fraction in males, consistent with our findings.

### Increased Cd4+ T-cell Memory Cells in Blood of Smokers

For a total of 10 studies we had smoking status information (FIG. 2A). Compared to age and sex, the number of associations were much lower (FIG. 3C). The most significant and consistent associations were displayed by the CD4+ T-cell memory fraction, which was significantly higher in smokers in at least 6 of the 10 studies. This finding is consistent with a study that performed blood cell-counts in 60 smokers & non-smokers. In 5 of the 10 studies, we also observed a consistent decrease in the NK-cell fraction in smokers, in line with reports of decreased cytotoxic NK counts in the blood of individuals afflicted with smoking-related diseases like cancer.

### CTH Adjustment at High Resolution Dampens Biological Age Associations

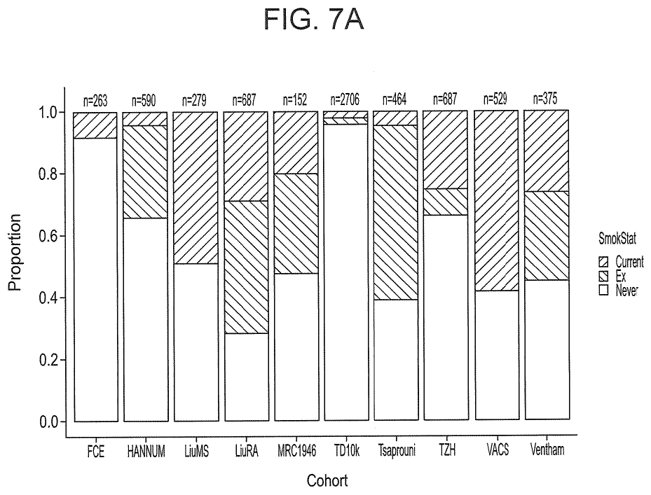

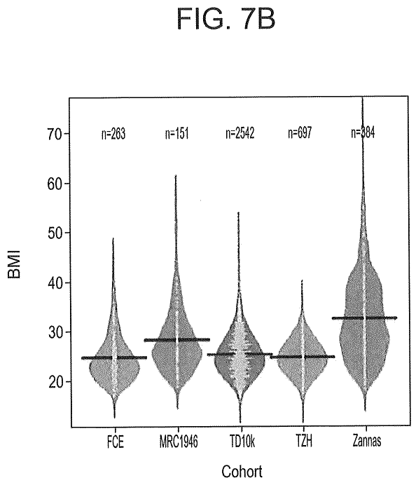

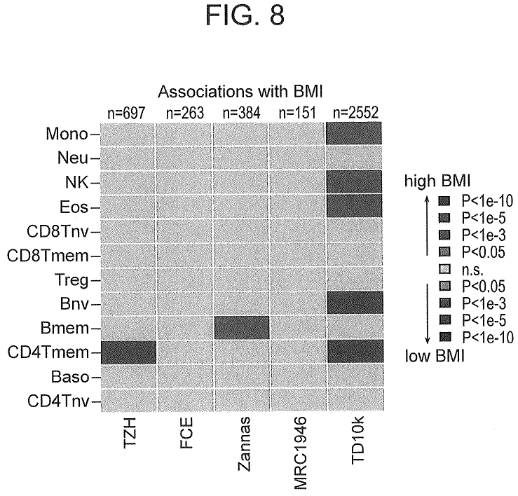

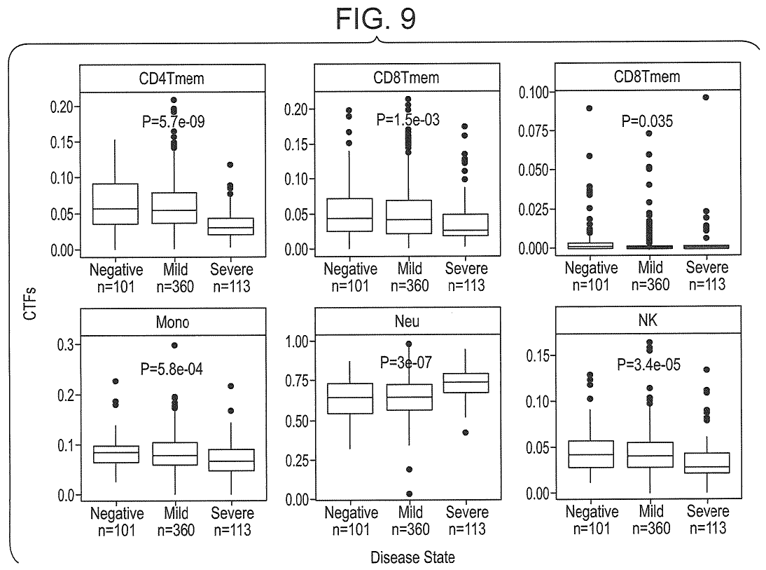

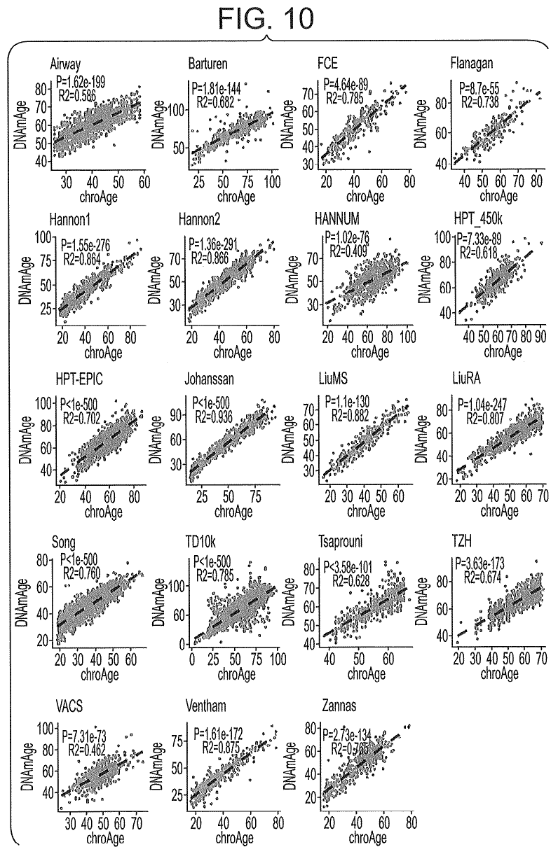

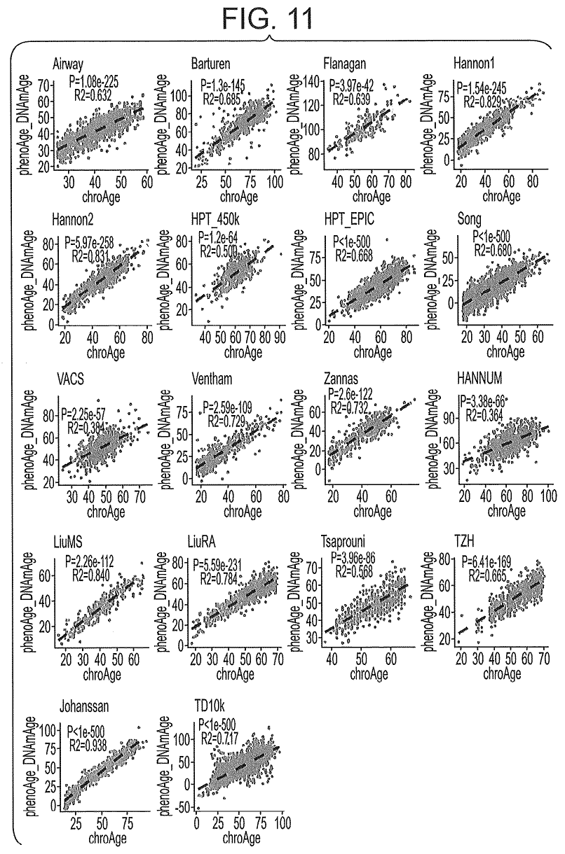

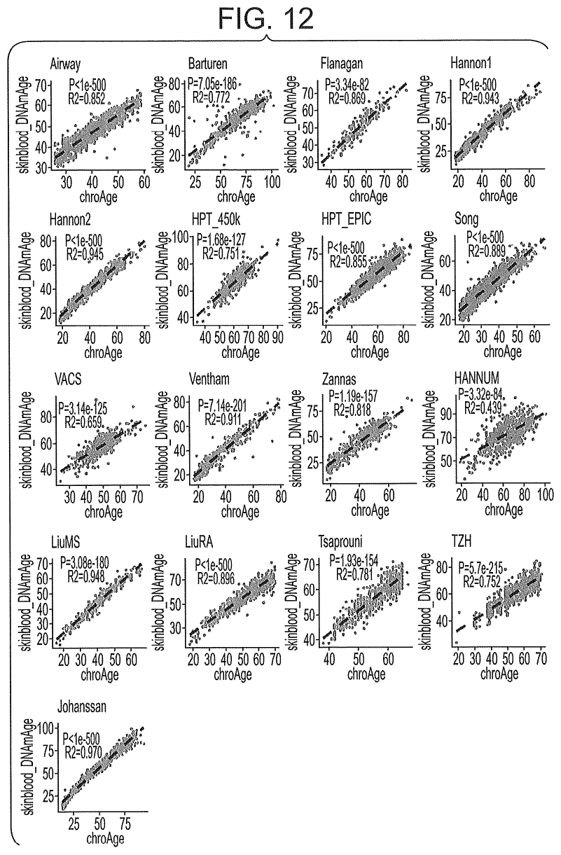

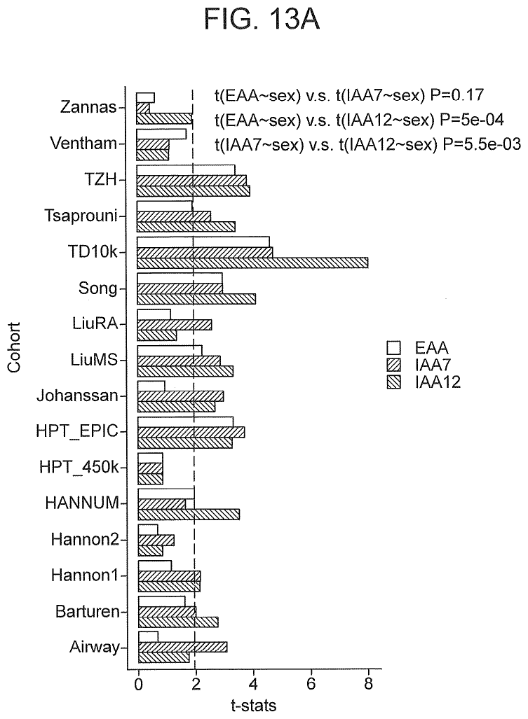

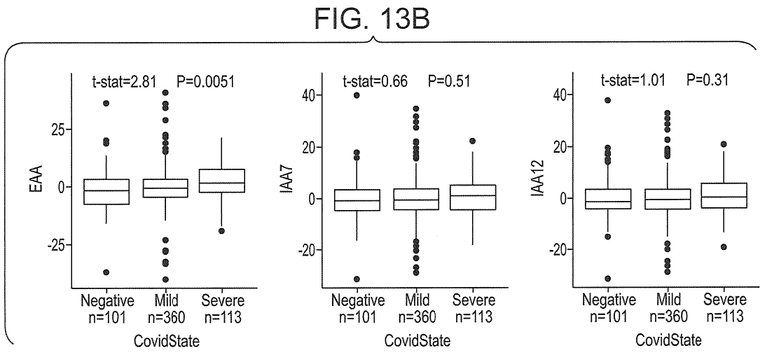

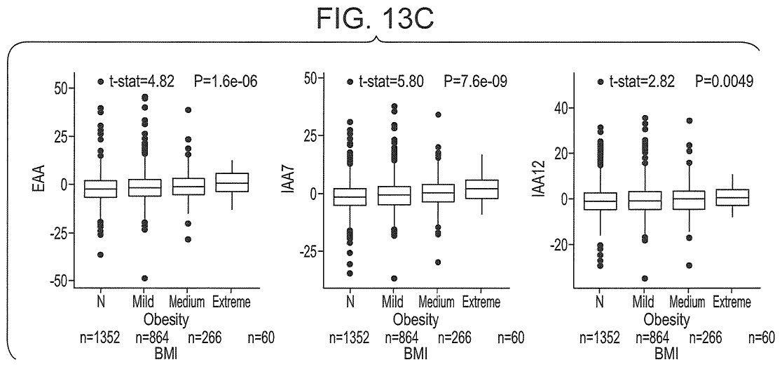

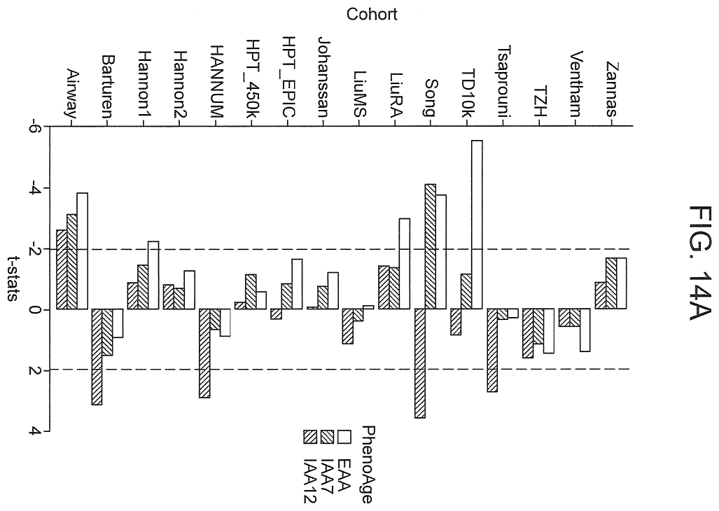

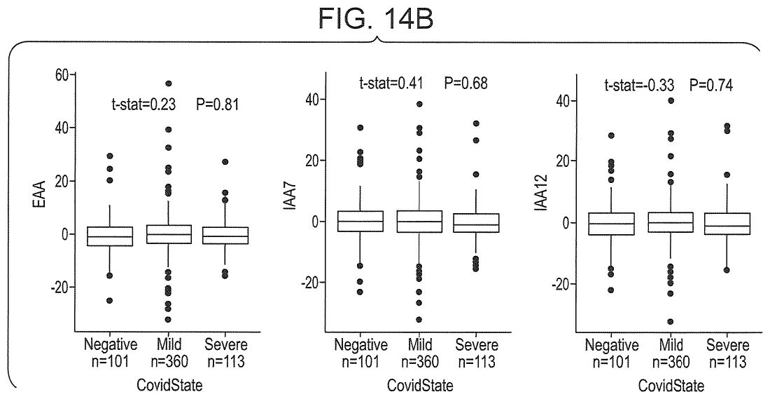

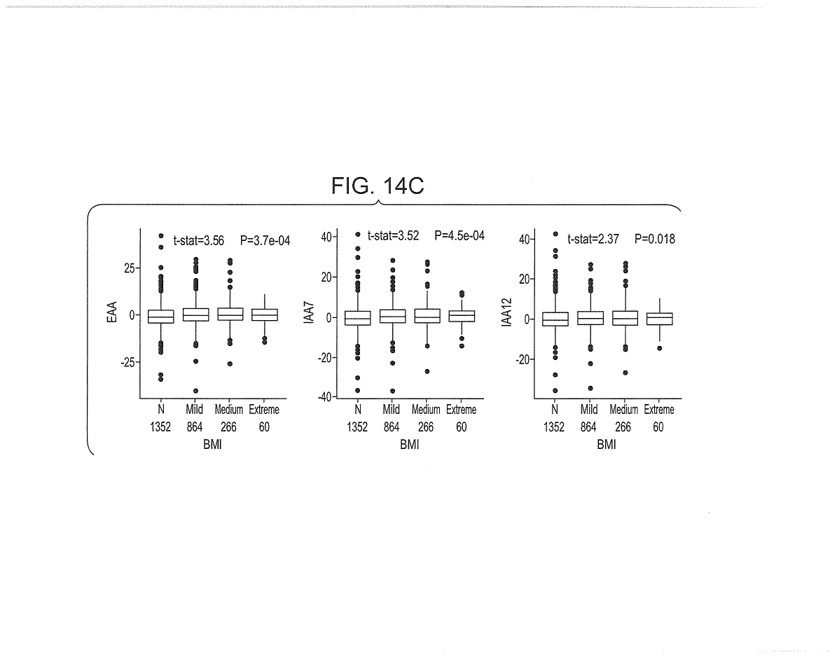

Next, we decided to explore how CTH affects associations of age-accelerations measures, as derived by epigenetic clocks, with key factors such as sex and smoking. Specifically, for each considered clock, we defined the extrinsic age-acceleration (EAA, unadjusted for CTH) and two different intrinsic age-acceleration (IAA) measures, one adjusted for the 7 main blood cell subtypes (IAA7) and another adjusted for 12 (IAA12). First, we verified that the DNAmAge as predicted with Horvath's multi-tissue, skin-blood and PhenoAge clocks correlated well with chronological age in all cohorts examined, with Horvath's multi-tissue clock displaying overall stronger correlations (FIGS. 7A, 7B, 8 and 10). Next, we computed the EAA and the two IAA measures and correlated these to sex. We observed cohorts where the significant association of EAA or IAA7 with sex (increased age-acceleration in males compared to females) disappeared once adjustment for 12 cell-types is made, but there were also cohorts where the association for IAA12 remained significant (FIG. 9). Overall, however, it is clear that adjustment of the age-acceleration residuals with 12 blood cell-type fractions led to a significant general dampening of the associations (FIG. 9). This highlights the importance of adjustment at high cellular resolution, but also indicates that the increased age-acceleration in males is also due to factors other than shifts in immune-cell fractions.

### Discussion

Among the novel insights contributed by this study, it is worth highlighting the following: an increased eosinophil count with age of childhood cancer survivors. This is significant because childhood cancer survivors are known to be at a much higher risk of developing heart disease, and one particular rare condition that can lead to a range of cardiovascular disease manifestations is eosinophilic myocarditis, a condition associated with elevated eosinophil counts. Thus, our finding leads to the hypothesis that increased age-associated cardiovascular disease risk in childhood cancer survivors could be driven in part by an age-associated increase in eosinophils, triggered by chemotherapy.

### METHODS

### Illumina DNA Methylation Datasets

Below we provide details of the data source and processing of each dataset. Each dataset profiled whole or peripheral blood samples with Illumina DNAm beadarrays (EPIC or 450k). Further details are available in (FIG. 2A).
LiuMS: The 450k dataset from Kular et al was obtained from the NCBI GEO website under the accession number GSE106648 (https://www.ncbi.nlm.nih.gov/geo/query/acc.cgi?acc=GSE106648). We downloaded the series matrix file which contained the data processed with detection P-values. We only retained probes with no missing values across all samples. This data was subsequently normalized with BMIQ, resulting in a normalized data matrix for 483,567 probes and 279 peripheral blood samples (140 multiple-sclerosis patients+139 controls).
Song: The EPIC dataset from Song et al profiled DNAm in blood from childhood cancer survivors and was obtained from the NCBI GEO website under the accession number GSE169156 (https://www.ncbi.nlm.nih.gov/geo/query/acc.cgi?acc=GSE169156). The file “GSE169156_RAW.tar” which contains the IDAT files was downloaded and processed with minfi package. Probes with P values <0.05 across all samples were retained. The filtered data was subsequently normalized with BMIQ, resulting in a normalized data matrix for 823,395 probes and 2052 samples.
HPT-EPIC & HPT-450k: These datasets derived DNAm profiles from the peripheral blood of African-Americans as part of The Genetic Epidemiology Network of Arteriopathy (GENOA) study. Data was obtained from the NCBI GEO websites under the accession numbers GSE210255 (https://www.ncbi.nlm.nih.gov/geo/query/acc.cgi?acc=GSE210255, Infinium HumanMethylationEPIC BeadChip) and GSE210254 (https://www.ncbi.nlm.nih.gov/geo/query/acc.cgi?acc=GSE210254, Infinium HumanMethylation450k BeadChip). The files “GSE210255_RAW.tar” and “GSE210254_RAW.tar” containing the IDAT files were downloaded and processed with minfi package. Probes with P values <0.05 across all samples were retained. The filtered data was subsequently normalized with BMIQ, resulting in normalized data matrices containing 826,512 probes and 1394 samples (EPIC set), and 476,722 probes and 418 samples (450k set), respectively.
Barturen: The EPIC dataset from Barturen et al profiled DNAm in blood from Covid-19 patients with three different levels of disease severity. Data was obtained from the NCBI GEO website under accession number GSE179325 (https://www.ncbi.nlm.nih.gov/geo/query/acc.cgi?acc=GSE179325). The file “GSE179325_RAW.tar” containing IDAT files was downloaded and processed with minfi package. Only probes with P values <0.05 across all samples were retained. The filtered data was subsequently normalized with BMIQ, resulting in a normalized data matrix for 845,921 probes and 574 samples.
Airwave: The EPIC dataset from the Airwave study was obtained from the NCBI GEO website under accession number GSE147740 (https://www.ncbi.nlm.nih.gov/geo/query/acc.cgi?acc=GSE147740). The file “GSE147740_RAW.tar” containing IDAT files was downloaded and processed with minfi. Only probes with P-values <0.05 across all samples were retained. Filtered data was subsequently normalized with BMIQ, resulting in a normalized data matrix for 840,034 probes and 1129 samples.
VACS: The 450k dataset from Zhang X et al was obtained from the NCBI GEO website under accession number GSE17860 (https://www.ncbi.nlm.nih.gov/geo/query/acc.cgi?acc-GSE117860). The file “GSE117860_MethylatedSignal.txt.gz” containing the unmethylated signals, methylated signals and detection P-values was downloaded. The beta values were obtained using the Illumina definition: methylated signal/(methylated signal+unmethylated signal+100). Only probes with P-values <0.05 across all samples were retained. The filtered beta value matrix was subsequently normalized with BMIQ, resulting in a normalized data matrix 396,327 probes across 529 samples.
Ventham: The 450k dataset from Ventham et al was obtained from NCBI GEO website under accession number GSE87648 (https://www.ncbi.nlm.nih.gov/geo/query/acc.cgi?acc=GSE87648). The file “GSE87648_RAW.tar” containing the IDAT files was downloaded and processed with minfi package. Two samples in which the proportion of probes with P-values <0.05 is lower than 0.99 were excluded, and probes with P-values <0.05 across all remaining samples were kept. Filtered data was subsequently normalized with BMIQ, resulting in a normalized data matrix for 470,807 probes and 382 samples.
Hannon-1 and 2: The 450k datasets from Hannon et al were obtained from NCBI GEO websites under accession numbers GSE80417 (https://www.ncbi.nlm.nih.gov/geo/query/acc.cgi?acc=GSE80417) and GSE84727 (https://www.ncbi.nlm.nih.gov/geo/query/acc.cgi?acc-GSE84727), which represent the phase-1 and phase-2 of their study. For the phase-1 dataset, the file “GSE80417_rawBetas.csv.gz” containing the beta values for filtered probes was downloaded, and the beta values were then normalized with BMIQ algorithm. For the phase-2 dataset, the file “GSE84727_rawBetas.csv.gz” and “GSE84727_detectionP.csv.gz” were downloaded. Only probes with P-values <0.05 across all samples were kept. The filtered beta value data was subsequently normalized with BMIQ. The final data matrices contain 477,818 probes across 675 samples, and 478,630 probes across 847 samples, for phase-1 and phase-2, respectively.
Zannas: This 450k dataset is derived from whole blood of African American participants of the Grady Trauma Project. Data was obtained from NCBI GEO website under accession number GSE72680 (https://www.ncbi.nlm.nih.gov/geo/query/acc.cgi?acc=GSE72680). The file “GSE72680_beta_values.txt.gz” containing the beta values and detection P-values was downloaded. Probes with P-values <0.05 across all samples were kept. However, the beta-value matrix of the retained probes still contained NAs and these were imputed with the function impute.knn (k=5) from the impute R-package. Beta values were later normalized with BMIQ, resulting in a normalized data matrix containing 453,310 probes across 422 samples.
Flanagan/FBS: The 450k dataset Flanagan et al is from the Breakthrough Generations Study and was obtained from NCBI GEO website under the accession number GSE61151 (https://www.ncbi.nlm.nih.gov/geo/query/acc.cgi?acc=GSE61151). The file “GSE61151_Matrix_raw_signal.txt.gz” containing beta values and detection P-values was downloaded. Only probes with P-values <0.05 and with no other QC-failures across all samples were kept. The filtered beta value data was subsequently normalized with BMIQ. The 2 duplicates of 4 pairs of samples were averaged, since duplicate pairs exhibited strongest correlations with each other. The final normalized data matrix was defined for 426,430 probes and 184 samples.
Johansson: The 450k dataset from Johansson et al was obtained from NCBI GEO website under accession number GSE87571 (https://www.ncbi.nlm.nih.gov/geo/query/acc.cgi?acc=GSE87571). The file “GSE87571_RAW.tar” containing the IDAT files was downloaded and processed with minfi package. Probes with P-values <0.05 across all samples were kept. Filtered data was subsequently normalized with BMIQ, resulting in a normalized data matrix for 475,069 probes across 732 samples.
TZH, Hannum, LiuRA, Tsaprouni, MRC1946 and FCE: The TZH (EPIC), Hannum (450k), LiuRA (450k), Tsaprouni (450k), MRC1946 (450k) and FCE (EPIC) datasets were downloaded and normalized as described by us previously.

### Real DNAm Datasets With Matched FACS Cell Counts

UCLA: UCLA Immune Assessment Core performed the analysis of immunosenescent cells for 144 whole blood samples, as described previously. Briefly, Total CD3+ T cells, CD4+ T cells, CD8+ T cells, CD19+ B cells, and CD56+/CD16+ NK cells were enumerated in EDTA whole blood with the BD Multitest 6-color TBNK reagent and BD Trucount tubes following the manufacturer's instructions, acquired on a BD FACSCanto II and analyzed with the BD FACSCanto Software. CD8+ T cell sub setting was performed by staining 50 μl of EDTA whole blood with CD3 FITC, CD8 PerCP, CD28 PE, and CD95 APC (BD) for 10 minutes, followed by BD FACS Lysing used according to the manufacturer's instructions. At least 10,000 lymphocyte events per sample were acquired and analyzed using DIVA 8.0 software on BD FACSCanto II.

We used the Illumina 450k dataset from Koestler et al consisting of 18 samples, of which 6 were whole blood (WB) and 12 were experimentally reconstructed “whole blood” mixtures. This dataset is available from GEO under accession number GSE77797. In the case of the 6 whole blood samples, flow-cytometric cell count estimates for the 6 major blood cell subtypes were available. For the experimental mixtures, the mixing proportions were determined by the experimentalist and therefore known without error. DNAm data was normalized and processed as previously described. In addition we analysed Illumina EPIC DNAm data for a total of 162 whole blood samples with matched FACS cell counts. The EPIC DNAm data was processed using minfi and BMIQ. This dataset is available from NODE under accession number (project ID) OEP000651, i.e. from https://www.biosino.org/node/project/detail/OEP000651

Data Availability: The two 12 blood cell-type DNAm reference matrices are available in SI tables S1 and S2, and are also part of the EpiDISH BioC R-package: http://www.bioconductor.org/packages/release/bioc/html/EpiDISH.html. Data analyzed in this manuscript is publicly available from GEO (www.ncbi.nlm.nih.gov/geo/) under accession numbers GSE

Code Availability: The two 12 blood cell-type DNAm reference matrices are available in SI tables S1 and S2, and have also been incorporated into the EpiDISH BioC R-package: http://www.bioconductor.org/packages/release/bioc/html/EpiDISH.html

Consistent with the above description, a new and improved method for determining percentages of twelve blood cell subtypes in a blood specimen includes steps of: (a) obtaining genomic DNA from the blood specimen of a human subject, (b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil, (c) comparing the unmethylated CG loci observed in (b) to unmethylated CG loci observed in genomic DNA collected from a reference group of human individuals and (d) correlating the unmethylated CG loci observed in (b) with the unmethylated CG loci observed in the reference group of human individuals to determine the percentage of twelve blood cell subtypes in the blood specimen.

In a first possible embodiment of the method, the CG loci are: cg21782213, cg10168351, cg03156020, cg17537714, cg13745692, cg04200580, cg15667493, cg21936454, cg15163862, cg19792189, cg10161198, cg19111287, cg05676874, cg03132824, cg17518643, cg09119854, cg11778706, cg04887735, cg22324641, cg08842268, cg09438865, cg08486432, cg04625250, cg08452021, cg20213624, cg02690807, cg22344918, cg18578898, cg19311567, cg16375820, cg08662013, cg11523325, cg21092682, cg23990235, cg24160371, cg01778017, cg05931551, cg03888645, cg21664107, cg06338239, cg01999608, cg08483099, cg23705316, cg11037382, cg11840035, cg21747310, cg20290061, cg07728865, cg19084822, cg03340102, cg03370360, cg04763150, cg11892665, cg21081729, cg24796422, cg05847670, cg00597139, cg12087639, cg24791846, cg01453386, cg14924828, cg17890852, cg07896398, cg00351957, cg25340205, cg02329886, cg21542081, cg06658453, cg08765214, cg11294011, cg03227316, cg23639055, cg25422758, cg04500050, cg15116227, cg09629758, cg02863748, cg03759549, cg25269402, cg08446606, cg19188904, cg09622285, cg26673070, cg04988216, cg21585800, cg09940675, cg14257017, cg15192986, cg14940779, cg17581877, cg11985063, cg05690666, cg20412398, cg19867897, cg23033014, cg03445419, cg17301153, cg04293820, cg14030593, cg26676468, cg03984633, cg25437005, cg19811231, cg26304744, cg27572854, cg22619531, cg09031281, cg00783080, cg00949794, cg09766370, cg27359629, cg05401393, cg08012294, cg04062032, cg11887733, cg03252427, cg12497611, cg06527889, cg13641221, cg12733242, cg18409707, cg02038353, cg12047375, cg03565732, cg18001026, cg23777479, cg15149205, cg22936341, cg07627628, cg04383721, cg05596912, cg02501773, cg15569739, cg24379742, cg00944599, cg15001636, cg13680246, cg12761472, cg19436514, cg00843117, cg17964575, cg26950756, cg27025274, cg16461337, cg13539891, cg23853980, cg07880190, cg26129027, cg15683542, cg10583651, cg05785919, cg22784669, cg18811564, cg03370491, cg18395354, cg11963313, cg26960322, cg10287321, cg27195778, cg04987479, cg02198280, cg09488807, cg07084675, cg00601124, cg27168830, cg25788647, cg02011162, cg22206842, cg20913547, cg24800373, cg15870556, cg04923194, cg14733637, cg17514643, cg03517570, cg07008893, cg14495399, cg03578212, cg26058722, cg23063536, cg19132501, cg03653880, cg24128943, cg10751207, cg10164437, cg12270000, cg07610771, cg27546582, cg00322928, cg26510178, cg21581865, cg05022030, cg22574467, cg01389234, cg26687322, cg19606289, cg06264696, cg20976977, cg14511575, cg05294459, cg25099065, cg14668581, cg10478600, cg15683107, cg07076850, cg15817752, cg07526731, cg05484949, cg13429555, cg26676273, cg06016162, cg02331158, cg16054240, cg26264767, cg08437936, cg07094228, cg27180005, cg07373578, cg13918228, cg02566312, cg09681537, cg17634650, cg25605180, cg00476430, cg18358155, cg01841368, cg04496578, cg02522485, cg16495530, cg07390370, cg25904998, cg05810879, cg24367295, cg19776833, cg14095525, cg16289232, cg05816398, cg02641941, cg26330304, cg04429341, cg11518040, cg03748473, cg04982372, cg27436324, cg06681449, cg25735823, cg06738507, cg10809134, cg11812782, cg25271915, cg05501327, cg22572158, cg04003505, cg22438885, cg12882261, cg23516026, cg23148481, cg25844471, cg10419892, cg15463664, cg08445080, cg15921515, cg24306984, cg23539484, cg03922997, cg05487307, cg27340845, cg03217619, cg13477627, cg15033776, cg15164173, cg08358686, cg08888548, cg00678912, cg15560912, cg07003611, cg16255804, cg07833643, cg26451108, cg08337454, cg15908108, cg17642929, cg03833097, cg18857045, cg23411838, cg00409021, cg17290867, cg16044562, cg02105817, cg18459351, cg05106785, cg10208609, cg16901161, cg02033323, cg24998002, cg07461115, cg04311057, cg06026449, cg00052246, cg02148547, cg02203103, cg20063728, cg20813776, cg13475329, cg13211380, cg20652042, cg10651616, cg02826841, cg05892024, cg25918166, cg03267512, cg27298253, cg18327204, cg19653890, cg02319946, cg18073938, cg21023071, cg07661702, cg16613029, cg13452923, cg11749308, cg10220401, cg08657911, cg17523148, cg24898812, cg04017239, cg22250168, cg11249120, cg04467549, cg01850245, cg11693925, cg04705866, cg24240349, cg01923140, cg12695059, cg05036909, cg05982017, cg26429965, cg09856271, cg08243981, cg14860676, cg22755579, cg05689028, cg15661018, cg05471842, cg00639010, cg19253785, cg16438469, cg03501128, cg04657000, cg15213052, cg24422171, cg19850895, cg10587583, cg05478809, cg25448766, cg22556056, cg03077364, cg18329839, cg07702659, cg17351376, cg13487687, cg07707056, cg20697519, cg04394724, cg12742224, cg00644351, cg13693582, cg23172664, cg16596052, cg14027234, cg01002223, cg04928784, cg16253157, cg12043508, cg16704246, cg04047276, cg16070350, cg20330224, cg02053685, cg00790439, cg26796793, cg20492070, cg12152193, cg16780406, cg06419846, cg18844901, cg19792099, cg14110920, cg21631387, cg20560862, cg17445453, cg02865554, cg07756788, cg09799856, cg17829179, cg25235205, cg11020586, cg20587232, cg04249416, cg19764973, cg24818060, cg06421734, cg11649969, cg04148640, cg01355027, cg15301710, cg01511347, cg17960717, cg00006459, cg15873326, cg11668148, cg09596645, cg08538069, cg02233915, cg22106847, cg07033790, cg05840984, cg10220104, cg17030380, cg02803925, cg14084609, cg16909341, cg05736642, cg13531735, cg01404879, cg21503771, cg15662508, cg05906696, cg09803743, cg12910830, cg15090899, cg22034357, cg17105176, cg24732344, cg15997319, cg09010228, cg11262246, cg05089849, cg22954836, cg02179849, cg25293601, cg24057263, cg01048814, cg08077807, cg00637085, cg17542165, cg13123707, cg26156120, cg18852698, cg18629096, cg27274718, cg05875239, cg13617280, cg24410690, cg05355684, cg18584272, cg18546155, cg26275360, cg14563201, cg08326410, cg02855870, cg15956469, cg27040968, cg10206039, cg09932794, cg09304968, cg00949861, cg02847139, cg25386954, cg04737115, cg03368758, cg14058851, cg23369683, cg14047092, cg27495654, cg22829007, cg16995933, cg01416168, cg08530578, cg07543798, cg12851731, cg15646529, cg03538296, cg13257250, cg17211761, cg18948661, cg13598219, cg22668643, cg09134152, cg21180201, cg08907176, cg03559700, cg21275838, cg05508074, cg18857340, cg12973514, cg16360310, cg24137511, cg16316183, cg25600606, cg18646784, cg05956380, cg16683571, cg18330536, cg17781418, cg13468144, cg26849760, cg23338668, cg13155300, cg24392753, cg22400420, cg10934870, cg14095610, cg01923724, cg06500418, cg04179951, cg16597887, cg04036920, cg15715903, cg01701207, cg08218512, cg07619820, cg01699630, cg25790531, cg19619956, cg26396370, cg05170732, cg08091050, cg09622624, cg02379348, cg25576801, cg27393431, cg09601944, cg24131359, cg26979142, cg04682832, cg25757820, cg26789411, cg17331920, cg00434123, cg16107565, cg23911433, cg12578084, cg15810023, cg08771884, cg07993518, cg20030158, cg20077668, cg27646850, cg15173892, cg07617198, cg10480329, cg20374292, cg00771778, cg18066690, cg02647842, cg20290017, cg02244028, cg06636137, cg25605773, cg22675702, cg08844422, cg26872907, cg23244761, cg23373670, cg14172797, cg26201751, cg02290460, cg18356337, cg00908794, cg08965078, cg16636767, cg09592164, cg17195809, cg19336573, cg03135975, cg05065075, cg01407558, cg08097676, cg23312369, cg11620863, cg10624395, cg03556458, cg14116399, cg19501306, cg02007480, cg23950707, cg14458725, cg14898005, cg01950767, cg18844042, cg05923857, cg01842756, cg03963853, cg11183587, cg21185441, cg02647748, cg14806960, and cg04851465.

In a second possible embodiment of the method, the CG loci are cg13745692, cg15667493, cg21936454, cg15163862, cg19111287, cg03132824, cg09119854, cg04887735, cg08486432, cg18578898, cg16375820, cg11523325, cg24160371, cg05931551, cg03888645, cg11840035, cg21747310, cg07728865, cg19084822, cg17894779, cg24503957, cg14138047, cg00694660, cg13484324, cg01364581, cg02725065, cg07133479, cg02280893, cg03069943, cg07643762, cg21200667, cg06828418, cg17824906, cg00378228, cg24463664, cg18984002, cg10091155, cg09177577, cg11840104, cg15958839, cg16192197, cg13104185, cg15009294, cg00629382, cg00705661, cg00208274, cg24211304, cg20558320, cg04073934, cg05158109, cg21081729, cg12087639, cg24791846, cg02329886, cg11294011, cg23639055, cg25422758, cg09622285, cg26673070, cg04988216, cg09940675, cg15192986, cg23033014, cg14030593, cg26676468, cg19699264, cg17170133, cg12037509, cg03380342, cg24736010, cg17436460, cg18469148, cg24496563, cg21039188, cg05506809, cg00712106, cg12646067, cg03184452, cg07862744, cg14633252, cg17282428, cg02587120, cg00551398, cg26009797, cg00086283, cg09275783, cg15106420, cg22197708, cg15003763, cg01782059, cg13894852, cg09131332, cg07188551, cg26252958, cg06706029, cg13394713, cg26603598, cg05201777, cg11809342, cg23743708, cg25437005, cg19811231, cg26304744, cg27572854, cg27359629, cg08012294, cg11887733, cg12047375, cg23777479, cg15149205, cg07627628, cg24379742, cg00944599, cg15001636, cg13680246, cg12761472, cg00843117, cg17964575, cg26950756, cg07880190, cg26129027, cg15683542, cg10583651, cg18289508, cg23005797, cg07784293, cg09222732, cg16497398, cg02619107, cg12419766, cg07340499, cg06828613, cg23635663, cg23506728, cg27297043, cg24525744, cg06288253, cg25404597, cg21814995, cg00292305, cg19622138, cg18303608, cg27518047, cg18835815, cg18051361, cg10295609, cg05519606, cg23271530, cg13011976, cg09449150, cg18395354, cg26960322, cg10287321, cg00601124, cg04923194, cg14733637, cg14495399, cg03653880, cg24128943, cg26510178, cg20976977, cg07316730, cg00148025, cg25382573, cg15829423, cg08220696, cg26383838, cg22084515, cg07960360, cg09241714, cg08761324, cg01884715, cg07044414, cg03680932, cg06369076, cg04330057, cg00083596, cg27576271, cg13678009, cg14101687, cg20561261, cg27157619, cg23900696, cg12208691, cg18822703, cg04329659, cg13551330, cg03822267, cg27416848, cg00087511, cg19277389, cg03717570, cg06099459, cg06541733, cg23731501, cg02079700, cg06419212, cg18861762, cg27317694, cg06013895, cg25099065, cg07076850, cg05484949, cg13429555, cg07373578, cg13918228, cg17634650, cg00476430, cg02522485, cg19776833, cg05816398, cg02641941, cg26330304, cg27436324, cg25735823, cg10809134, cg27184438, cg10361659, cg03653817, cg03328673, cg17453767, cg08246318, cg00649632, cg13276502, cg20847766, cg03386536, cg01598421, cg14422093, cg16098726, cg03238899, cg08986727, cg00374717, cg00863309, cg10678215, cg14905514, cg15552491, cg04074908, cg25107522, cg07433769, cg16586807, cg02563364, cg11972721, cg12796332, cg22528123, cg16417374, cg23325384, cg06916059, cg09299082, cg05776861, cg01502811, cg05501327, cg22572158, cg23516026, cg25844471, cg08445080, cg15921515, cg03922997, cg15033776, cg07003611, cg16255804, cg03833097, cg05106785, cg10208609, cg02033323, cg00052246, cg02148547, cg26408382, cg08562111, cg22360028, cg09490124, cg09197075, cg18124517, cg05878535, cg23519969, cg17498667, cg00463840, cg09642378, cg02397552, cg19876232, cg12485044, cg13345122, cg17392230, cg01554304, cg25557227, cg23541811, cg03873220, cg16685586, cg02979010, cg06840801, cg01865118, cg01436550, cg11812071, cg16637690, cg22980427, cg22577252, cg07805771, cg24599709, cg02272851, cg24847035, cg07793163, cg20063728, cg20652042, cg05892024, cg25918166, cg16613029, cg13452923, cg11249120, cg04467549, cg01850245, cg04705866, cg24240349, cg12695059, cg05982017, cg09856271, cg22755579, cg05689028, cg05471842, cg00639010, cg03501128, cg04657000, cg24037746, cg00460916, cg24618739, cg09736286, cg12983578, cg20417024, cg23320693, cg13805183, cg08035195, cg09096234, cg11161150, cg17542594, cg10335493, cg14776168, cg02763540, cg16946840, cg03315940, cg01356867, cg02586198, cg25503323, cg12524168, cg26509305, cg07109551, cg02205013, cg19884600, cg09088625, cg03534847, cg08877257, cg12159213, cg05905180, cg24422171, cg10587583, cg05478809, cg22556056, cg07702659, cg17351376, cg07707056, cg20697519, cg04394724, cg00644351, cg13693582, cg23172664, cg16596052, cg14027234, cg01002223, cg16253157, cg12043508, cg16704246, cg12152193, cg06419846, cg19792099, cg14110920, cg20560862, cg07756788, cg25235205, cg01949403, cg14507427, cg05496363, cg09872088, cg23463186, cg14468508, cg20367218, cg12221970, cg26856443, cg09628822, cg12655059, cg08585614, cg26668000, cg16359142, cg11067179, cg09127592, cg10365743, cg19556572, cg09586583, cg22213242, cg03788408, cg06748978, cg26354221, cg14537031, cg20681184, cg19764973, cg17960717, cg00006459, cg11668148, cg09596645, cg22106847, cg07033790, cg02803925, cg14084609, cg16909341, cg05736642, cg13531735, cg12910830, cg15090899, cg24732344, cg08077807, cg00637085, cg26156120, cg21237481, cg23153546, cg25203627, cg18550847, cg04538585, cg06680065, cg00391067, cg11197258, cg16362140, cg06315149, cg00170714, cg07410597, cg08061334, cg14209186, cg25578728, cg14697334, cg27000590, cg25381747, cg24706564, cg20315954, cg07766916, cg17732216, cg19788934, cg08969102, cg15427040, cg17738684, cg19968199, cg24459209, cg20240243, cg16472853, cg07201450, cg01820584, cg27274718, cg05875239, cg13617280, cg24410690, cg05355684, cg26275360, cg08326410, cg15956469, cg27040968, cg10206039, cg09304968, cg25386954, cg03368758, cg14058851, cg01416168, cg03538296, cg17211761, cg21180201, cg21275838, cg16360310, cg24137511, cg16316183, cg04992852, cg25846372, cg07802362, cg13254203, cg01112784, cg05253716, cg15705930, cg18087143, cg23001370, cg07873128, cg10830795, cg03891962, cg09278623, cg03055021, cg25029657, cg17169982, cg10157558, cg00515905, cg23617121, cg27309871, cg21777154, cg07123481, cg22917487, cg16220743, cg03956353, cg04718702, cg01830256, cg11790417, cg25600606, cg13468144, cg23338668, cg22400420, cg10934870, cg01923724, cg04036920, cg01699630, cg25790531, cg19619956, cg26396370, cg08091050, cg24131359, cg25757820, cg17331920, cg23911433, cg09694051, cg13633625, cg17078883, cg17322118, cg07079231, cg01632240, cg13785123, cg02969426, cg17567562, cg15457217, cg15169266, cg07052231, cg16102040, cg11525409, cg13618969, cg00591421, cg09166556, cg06494464, cg07804973, cg25693317, cg13589463, cg21772826, cg11809958, cg02279108, cg06784232, cg05492904, cg14523804, cg14249174, cg05006942, cg18619616, cg25539505, cg09948350, cg27466532, cg17547928, cg10480329, cg18066690, cg02244028, cg22675702, cg26872907, cg23244761, cg16636767, cg10624395, cg05923857, cg03963853, cg04851465, cg21459713, cg05242065, cg08908131, cg27366072, cg19301114, cg24788483, cg07002540, cg26124526, cg01041239, cg18589858, cg07125725, cg12655112, cg16356013, cg25376491, cg25898577, cg20641531, cg14043316, cg05452742, cg16728323, cg08694574, cg23719713, cg00646216, cg13290254, cg25517015, cg16766036, cg20918393, cg25291653, cg07505901, cg03567939, cg25765315, cg14872074, cg18770149, cg23391288, cg21983607, cg12037947, cg26953469, cg01024962, cg02780988, and cg14428166.

In a third possible embodiment of the method, the CG loci are cg13745692, cg15667493, cg21936454, cg15163862, cg19111287, cg03132824, cg09119854, cg04887735, cg08486432, cg18578898, cg16375820, cg11523325, cg24160371, cg05931551, cg03888645, cg11840035, cg21747310, cg07728865, cg19084822, cg21081729, cg12087639, cg24791846, cg02329886, cg11294011, cg23639055, cg25422758, cg09622285, cg26673070, cg04988216, cg09940675, cg15192986, cg23033014, cg14030593, cg26676468, cg25437005, cg19811231, cg26304744, cg27572854, cg27359629, cg08012294, cg11887733, cg12047375, cg23777479, cg15149205, cg07627628, cg24379742, cg00944599, cg15001636, cg13680246, cg12761472, cg00843117, cg17964575, cg26950756, cg07880190, cg26129027, cg15683542, cg10583651, cg18395354, cg26960322, cg10287321, cg00601124, cg04923194, cg14733637, cg14495399, cg03653880, cg24128943, cg26510178, cg20976977, cg25099065, cg07076850, cg05484949, cg13429555, cg07373578, cg13918228, cg17634650, cg00476430, cg02522485, cg19776833, cg05816398, cg02641941, cg26330304, cg27436324, cg25735823, cg10809134, cg05501327, cg22572158, cg23516026, cg25844471, cg08445080, cg15921515, cg03922997, cg15033776, cg07003611, cg16255804, cg03833097, cg05106785, cg10208609, cg02033323, cg00052246, cg02148547, cg20063728, cg20652042, cg05892024, cg25918166, cg16613029, cg13452923, cg11249120, cg04467549, cg01850245, cg04705866, cg24240349, cg12695059, cg05982017, cg09856271, cg22755579, cg05689028, cg05471842, cg00639010, cg03501128, cg04657000, cg24422171, cg10587583, cg05478809, cg22556056, cg07702659, cg17351376, cg07707056, cg20697519, cg04394724, cg00644351, cg13693582, cg23172664, cg16596052, cg14027234, cg01002223, cg16253157, cg12043508, cg16704246, cg12152193, cg06419846, cg19792099, cg14110920, cg20560862, cg07756788, cg25235205, cg19764973, cg17960717, cg00006459, cg11668148, cg09596645, cg22106847, cg07033790, cg02803925, cg14084609, cg16909341, cg05736642, cg13531735, cg12910830, cg15090899, cg24732344, cg08077807, cg00637085, cg26156120, cg27274718, cg05875239, cg13617280, cg24410690, cg05355684, cg26275360, cg08326410, cg15956469, cg27040968, cg10206039, cg09304968, cg25386954, cg03368758, cg14058851, cg01416168, cg03538296, cg17211761, cg21180201, cg21275838, cg16360310, cg24137511, cg16316183, cg25600606, cg13468144, cg23338668, cg22400420, cg10934870, cg01923724, cg04036920, cg01699630, cg25790531, cg19619956, cg26396370, cg08091050, cg24131359, cg25757820, cg17331920, cg23911433, cg10480329, cg18066690, cg02244028, cg22675702, cg26872907, cg23244761, cg16636767, cg10624395, cg05923857, cg03963853, and cg04851465.

The obtained genomic DNA from the blood specimen is derived from a human subject. Similarly, the genomic DNA obtained from the reference group of specimens is derived from a reference group of human individuals. Any number of different blood collection kits known in the art may be used to collect the blood of the human subject. In one possible embodiment, the human subject is instructed to fast for a minimum of one hour prior to collecting the blood specimen.

The bisulfate conversion process referenced in step (b) is known in the art. Kits useful for bisulfate conversion are commercially available from a number of manufacturers including Human Genetic Signatures'Methyleasy and Chemicon's CpGenome Modification Kit. See also, WO04096825A1, which describes bisulfite modification methods and Olek et al. Nuc. Acids Res. 24:5064-6 (1994), which discloses methods of performing bisulfite treatment and subsequent amplification.

The performing of the bisulfate conversion process may include the steps of amplifying the genomic DNA of the blood specimen by a polymerase chain reaction process, hybridizing the genomic DNA to a beadchip to create a genomic DNA hybridized beadchip, staining the genomic DNA hybridized beadchip and then imaging the genomic DNA hybridized beadchip.

The imaging of the genomic DNA hybridized beadchip may be performed using the Illumina EPIC450k array which provides for unambiguous CG loci identification for purposes of observing methylation at specific CG loci of the human DNA genome. Alternatively, other methods of epigenetic beta value collection may be used if desired.

Still further, the method may include using a regression algorithm to correlate the CG loci methylation observed in (b) with the CG loci methylation observed in the reference group of blood specimens of known cell-type percentages. This allows for good correlation between the CG loci methylation observed in (b) with the CG loci methylation observed in the reference group of blood specimens resulting in a better or more accurate determination of the percentage of cell-types in a blood specimen.

This disclosure relates to the following items.
1. A method of determining percentages of twelve blood cell subtypes in a blood specimen, comprising:
   (a) obtaining genomic DNA from the blood specimen of a human subject;
   (b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil;
   (c) comparing the unmethylated CG loci observed in (b) to unmethylated CG loci observed in genomic DNA collected from a reference group of human individuals; and
   (d) correlating the unmethylated CG loci observed in (b) with the unmethylated CG loci observed in the reference group of human individuals to determine the percentage of the twelve blood cell subtypes in the blood specimen wherein the CG loci are cg21782213, cg10168351, cg03156020, cg17537714, cg13745692, cg04200580, cg15667493, cg21936454, cg15163862, cg19792189, cg10161198, cg19111287, cg05676874, cg03132824, cg17518643, cg09119854, cg11778706, cg04887735, cg22324641, cg08842268, cg09438865, cg08486432, cg04625250, cg08452021, cg20213624, cg02690807, cg22344918, cg18578898, cg19311567, cg16375820, cg08662013, cg11523325, cg21092682, cg23990235, cg24160371, cg01778017, cg05931551, cg03888645, cg21664107, cg06338239, cg01999608, cg08483099, cg23705316, cg11037382, cg11840035, cg21747310, cg20290061, cg07728865, cg19084822, cg03340102, cg03370360, cg04763150, cg11892665, cg21081729, cg24796422, cg05847670, cg00597139, cg12087639, cg24791846, cg01453386, cg14924828, cg17890852, cg07896398, cg00351957, cg25340205, cg02329886, cg21542081, cg06658453, cg08765214, cg11294011, cg03227316, cg23639055, cg25422758, cg04500050, cg15116227, cg09629758, cg02863748, cg03759549, cg25269402, cg08446606, cg19188904, cg09622285, cg26673070, cg04988216, cg21585800, cg09940675, cg14257017, cg15192986, cg14940779, cg17581877, cg11985063, cg05690666, cg20412398, cg19867897, cg23033014, cg03445419, cg17301153, cg04293820, cg14030593, cg26676468, cg03984633, cg25437005, cg19811231, cg26304744, cg27572854, cg22619531, cg09031281, cg00783080, cg00949794, cg09766370, cg27359629, cg05401393, cg08012294, cg04062032, cg11887733, cg03252427, cg12497611, cg06527889, cg13641221, cg12733242, cg18409707, cg02038353, cg12047375, cg03565732, cg18001026, cg23777479, cg15149205, cg22936341, cg07627628, cg04383721, cg05596912, cg02501773, cg15569739, cg24379742, cg00944599, cg15001636, cg13680246, cg12761472, cg19436514, cg00843117, cg17964575, cg26950756, cg27025274, cg16461337, cg13539891, cg23853980, cg07880190, cg26129027, cg15683542, cg10583651, cg05785919, cg22784669, cg18811564, cg03370491, cg18395354, cg11963313, cg26960322, cg10287321, cg27195778, cg04987479, cg02198280, cg09488807, cg07084675, cg00601124, cg27168830, cg25788647, cg02011162, cg22206842, cg20913547, cg24800373, cg15870556, cg04923194, cg14733637, cg17514643, cg03517570, cg07008893, cg14495399, cg03578212, cg26058722, cg23063536, cg19132501, cg03653880, cg24128943, cg10751207, cg10164437, cg12270000, cg07610771, cg27546582, cg00322928, cg26510178, cg21581865, cg05022030, cg22574467, cg01389234, cg26687322, cg19606289, cg06264696, cg20976977, cg14511575, cg05294459, cg25099065, cg14668581, cg10478600, cg15683107, cg07076850, cg15817752, cg07526731, cg05484949, cg13429555, cg26676273, cg06016162, cg02331158, cg16054240, cg26264767, cg08437936, cg07094228, cg27180005, cg07373578, cg13918228, cg02566312, cg09681537, cg17634650, cg25605180, cg00476430, cg18358155, cg01841368, cg04496578, cg02522485, cg16495530, cg07390370, cg25904998, cg05810879, cg24367295, cg19776833, cg14095525, cg16289232, cg05816398, cg02641941, cg26330304, cg04429341, cg11518040, cg03748473, cg04982372, cg27436324, cg06681449, cg25735823, cg06738507, cg10809134, cg11812782, cg25271915, cg05501327, cg22572158, cg04003505, cg22438885, cg12882261, cg23516026, cg23148481, cg25844471, cg10419892, cg15463664, cg08445080, cg15921515, cg24306984, cg23539484, cg03922997, cg05487307, cg27340845, cg03217619, cg13477627, cg15033776, cg15164173, cg08358686, cg08888548, cg00678912, cg15560912, cg07003611, cg16255804, cg07833643, cg26451108, cg08337454, cg15908108, cg17642929, cg03833097, cg18857045, cg23411838, cg00409021, cg17290867, cg16044562, cg02105817, cg18459351, cg05106785, cg10208609, cg16901161, cg02033323, cg24998002, cg07461115, cg04311057, cg06026449, cg00052246, cg02148547, cg02203103, cg20063728, cg20813776, cg13475329, cg13211380, cg20652042, cg10651616, cg02826841, cg05892024, cg25918166, cg03267512, cg27298253, cg18327204, cg19653890, cg02319946, cg18073938, cg21023071, cg07661702, cg16613029, cg13452923, cg11749308, cg10220401, cg08657911, cg17523148, cg24898812, cg04017239, cg22250168, cg11249120, cg04467549, cg01850245, cg11693925, cg04705866, cg24240349, cg01923140, cg12695059, cg05036909, cg05982017, cg26429965, cg09856271, cg08243981, cg14860676, cg22755579, cg05689028, cg15661018, cg05471842, cg00639010, cg19253785, cg16438469, cg03501128, cg04657000, cg15213052, cg24422171, cg19850895, cg10587583, cg05478809, cg25448766, cg22556056, cg03077364, cg18329839, cg07702659, cg17351376, cg13487687, cg07707056, cg20697519, cg04394724, cg12742224, cg00644351, cg13693582, cg23172664, cg16596052, cg14027234, cg01002223, cg04928784, cg16253157, cg12043508, cg16704246, cg04047276, cg16070350, cg20330224, cg02053685, cg00790439, cg26796793, cg20492070, cg12152193, cg16780406, cg06419846, cg18844901, cg19792099, cg14110920, cg21631387, cg20560862, cg17445453, cg02865554, cg07756788, cg09799856, cg17829179, cg25235205, cg11020586, cg20587232, cg04249416, cg19764973, cg24818060, cg06421734, cg11649969, cg04148640, cg01355027, cg15301710, cg01511347, cg17960717, cg00006459, cg15873326, cg11668148, cg09596645, cg08538069, cg02233915, cg22106847, cg07033790, cg05840984, cg10220104, cg17030380, cg02803925, cg14084609, cg16909341, cg05736642, cg13531735, cg01404879, cg21503771, cg15662508, cg05906696, cg09803743, cg12910830, cg15090899, cg22034357, cg17105176, cg24732344, cg15997319, cg09010228, cg11262246, cg05089849, cg22954836, cg02179849, cg25293601, cg24057263, cg01048814, cg08077807, cg00637085, cg17542165, cg13123707, cg26156120, cg18852698, cg18629096, cg27274718, cg05875239, cg13617280, cg24410690, cg05355684, cg18584272, cg18546155, cg26275360, cg14563201, cg08326410, cg02855870, cg15956469, cg27040968, cg10206039, cg09932794, cg09304968, cg00949861, cg02847139, cg25386954, cg04737115, cg03368758, cg14058851, cg23369683, cg14047092, cg27495654, cg22829007, cg16995933, cg01416168, cg08530578, cg07543798, cg12851731, cg15646529, cg03538296, cg13257250, cg17211761, cg18948661, cg13598219, cg22668643, cg09134152, cg21180201, cg08907176, cg03559700, cg21275838, cg05508074, cg18857340, cg12973514, cg16360310, cg24137511, cg16316183, cg25600606, cg18646784, cg05956380, cg16683571, cg18330536, cg17781418, cg13468144, cg26849760, cg23338668, cg13155300, cg24392753, cg22400420, cg10934870, cg14095610, cg01923724, cg06500418, cg04179951, cg16597887, cg04036920, cg15715903, cg01701207, cg08218512, cg07619820, cg01699630, cg25790531, cg19619956, cg26396370, cg05170732, cg08091050, cg09622624, cg02379348, cg25576801, cg27393431, cg09601944, cg24131359, cg26979142, cg04682832, cg25757820, cg26789411, cg17331920, cg00434123, cg16107565, cg23911433, cg12578084, cg15810023, cg08771884, cg07993518, cg20030158, cg20077668, cg27646850, cg15173892, cg07617198, cg10480329, cg20374292, cg00771778, cg18066690, cg02647842, cg20290017, cg02244028, cg06636137, cg25605773, cg22675702, cg08844422, cg26872907, cg23244761, cg23373670, cg14172797, cg26201751, cg02290460, cg18356337, cg00908794, cg08965078, cg16636767, cg09592164, cg17195809, cg19336573, cg03135975, cg05065075, cg01407558, cg08097676, cg23312369, cg11620863, cg10624395, cg03556458, cg14116399, cg19501306, cg02007480, cg23950707, cg14458725, cg14898005, cg01950767, cg18844042, cg05923857, cg01842756, cg03963853, cg11183587, cg21185441, cg02647748, cg14806960, and cg04851465.
2. The method of item 1, including amplifying the genomic DNA of the blood specimen by a polymerase chain reaction.
3. The method of item 2, wherein the observing of the unmethylated CG loci, in the genomic DNA of the human subject includes hybridizing the genomic DNA to a beadchip to create a genomic DNA hybridized beadchip and then imaging the genomic DNA hybridized beadchip.
4. The method of item 3, including using a regression algorithm to correlate the unmethylated CG loci observed in (b) with the unmethylated GC loci observed in the reference group of human individuals.
5. The method of item 1, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.
6. A method of determining percentage of twelve blood cell subtypes in a blood specimen, comprising:
   (a) obtaining genomic DNA from the blood specimen of a human subject;
   (b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil;
   (c) comparing the unmethylated CG loci observed in (b) to unmethylated CG loci observed in genomic DNA collected from a reference group of human individuals; and
   (d) correlating the unmethylated CG loci observed in (b) with the unmethylated CG loci observed in the reference group of human individuals to determine the percentage of the twelve blood cell subtypes in the blood specimen wherein the CG loci are cg13745692, cg15667493, cg21936454, cg15163862, cg19111287, cg03132824, cg09119854, cg04887735, cg08486432, cg18578898, cg16375820, cg11523325, cg24160371, cg05931551, cg03888645, cg11840035, cg21747310, cg07728865, cg19084822, cg17894779, cg24503957, cg14138047, cg00694660, cg13484324, cg01364581, cg02725065, cg07133479, cg02280893, cg03069943, cg07643762, cg21200667, cg06828418, cg17824906, cg00378228, cg24463664, cg18984002, cg10091155, cg09177577, cg11840104, cg15958839, cg16192197, cg13104185, cg15009294, cg00629382, cg00705661, cg00208274, cg24211304, cg20558320, cg04073934, cg05158109, cg21081729, cg12087639, cg24791846, cg02329886, cg11294011, cg23639055, cg25422758, cg09622285, cg26673070, cg04988216, cg09940675, cg15192986, cg23033014, cg14030593, cg26676468, cg19699264, cg17170133, cg12037509, cg03380342, cg24736010, cg17436460, cg18469148, cg24496563, cg21039188, cg05506809, cg00712106, cg12646067, cg03184452, cg07862744, cg14633252, cg17282428, cg02587120, cg00551398, cg26009797, cg00086283, cg09275783, cg15106420, cg22197708, cg15003763, cg01782059, cg13894852, cg09131332, cg07188551, cg26252958, cg06706029, cg13394713, cg26603598, cg05201777, cg11809342, cg23743708, cg25437005, cg19811231, cg26304744, cg27572854, cg27359629, cg08012294, cg11887733, cg12047375, cg23777479, cg15149205, cg07627628, cg24379742, cg00944599, cg15001636, cg13680246, cg12761472, cg00843117, cg17964575, cg26950756, cg07880190, cg26129027, cg15683542, cg10583651, cg18289508, cg23005797, cg07784293, cg09222732, cg16497398, cg02619107, cg12419766, cg07340499, cg06828613, cg23635663, cg23506728, cg27297043, cg24525744, cg06288253, cg25404597, cg21814995, cg00292305, cg19622138, cg18303608, cg27518047, cg18835815, cg18051361, cg10295609, cg05519606, cg23271530, cg13011976, cg09449150, cg18395354, cg26960322, cg10287321, cg00601124, cg04923194, cg14733637, cg14495399, cg03653880, cg24128943, cg26510178, cg20976977, cg07316730, cg00148025, cg25382573, cg15829423, cg08220696, cg26383838, cg22084515, cg07960360, cg09241714, cg08761324, cg01884715, cg07044414, cg03680932, cg06369076, cg04330057, cg00083596, cg27576271, cg13678009, cg14101687, cg20561261, cg27157619, cg23900696, cg12208691, cg18822703, cg04329659, cg13551330, cg03822267, cg27416848, cg00087511, cg19277389, cg03717570, cg06099459, cg06541733, cg23731501, cg02079700, cg06419212, cg18861762, cg27317694, cg06013895, cg25099065, cg07076850, cg05484949, cg13429555, cg07373578, cg13918228, cg17634650, cg00476430, cg02522485, cg19776833, cg05816398, cg02641941, cg26330304, cg27436324, cg25735823, cg10809134, cg27184438, cg10361659, cg03653817, cg03328673, cg17453767, cg08246318, cg00649632, cg13276502, cg20847766, cg03386536, cg01598421, cg14422093, cg16098726, cg03238899, cg08986727, cg00374717, cg00863309, cg10678215, cg14905514, cg15552491, cg04074908, cg25107522, cg07433769, cg16586807, cg02563364, cg11972721, cg12796332, cg22528123, cg16417374, cg23325384, cg06916059, cg09299082, cg05776861, cg01502811, cg05501327, cg22572158, cg23516026, cg25844471, cg08445080, cg15921515, cg03922997, cg15033776, cg07003611, cg16255804, cg03833097, cg05106785, cg10208609, cg02033323, cg00052246, cg02148547, cg26408382, cg08562111, cg22360028, cg09490124, cg09197075, cg18124517, cg05878535, cg23519969, cg17498667, cg00463840, cg09642378, cg02397552, cg19876232, cg12485044, cg13345122, cg17392230, cg01554304, cg25557227, cg23541811, cg03873220, cg16685586, cg02979010, cg06840801, cg01865118, cg01436550, cg11812071, cg16637690, cg22980427, cg22577252, cg07805771, cg24599709, cg02272851, cg24847035, cg07793163, cg20063728, cg20652042, cg05892024, cg25918166, cg16613029, cg13452923, cg11249120, cg04467549, cg01850245, cg04705866, cg24240349, cg12695059, cg05982017, cg09856271, cg22755579, cg05689028, cg05471842, cg00639010, cg03501128, cg04657000, cg24037746, cg00460916, cg24618739, cg09736286, cg12983578, cg20417024, cg23320693, cg13805183, cg08035195, cg09096234, cg11161150, cg17542594, cg10335493, cg14776168, cg02763540, cg16946840, cg03315940, cg01356867, cg02586198, cg25503323, cg12524168, cg26509305, cg07109551, cg02205013, cg19884600, cg09088625, cg03534847, cg08877257, cg12159213, cg05905180, cg24422171, cg10587583, cg05478809, cg22556056, cg07702659, cg17351376, cg07707056, cg20697519, cg04394724, cg00644351, cg13693582, cg23172664, cg16596052, cg14027234, cg01002223, cg16253157, cg12043508, cg16704246, cg12152193, cg06419846, cg19792099, cg14110920, cg20560862, cg07756788, cg25235205, cg01949403, cg14507427, cg05496363, cg09872088, cg23463186, cg14468508, cg20367218, cg12221970, cg26856443, cg09628822, cg12655059, cg08585614, cg26668000, cg16359142, cg11067179, cg09127592, cg10365743, cg19556572, cg09586583, cg22213242, cg03788408, cg06748978, cg26354221, cg14537031, cg20681184, cg19764973, cg17960717, cg00006459, cg11668148, cg09596645, cg22106847, cg07033790, cg02803925, cg14084609, cg16909341, cg05736642, cg13531735, cg12910830, cg15090899, cg24732344, cg08077807, cg00637085, cg26156120, cg21237481, cg23153546, cg25203627, cg18550847, cg04538585, cg06680065, cg00391067, cg11197258, cg16362140, cg06315149, cg00170714, cg07410597, cg08061334, cg14209186, cg25578728, cg14697334, cg27000590, cg25381747, cg24706564, cg20315954, cg07766916, cg17732216, cg19788934, cg08969102, cg15427040, cg17738684, cg19968199, cg24459209, cg20240243, cg16472853, cg07201450, cg01820584, cg27274718, cg05875239, cg13617280, cg24410690, cg05355684, cg26275360, cg08326410, cg15956469, cg27040968, cg10206039, cg09304968, cg25386954, cg03368758, cg14058851, cg01416168, cg03538296, cg17211761, cg21180201, cg21275838, cg16360310, cg24137511, cg16316183, cg04992852, cg25846372, cg07802362, cg13254203, cg01112784, cg05253716, cg15705930, cg18087143, cg23001370, cg07873128, cg10830795, cg03891962, cg09278623, cg03055021, cg25029657, cg17169982, cg10157558, cg00515905, cg23617121, cg27309871, cg21777154, cg07123481, cg22917487, cg16220743, cg03956353, cg04718702, cg01830256, cg11790417, cg25600606, cg13468144, cg23338668, cg22400420, cg10934870, cg01923724, cg04036920, cg01699630, cg25790531, cg19619956, cg26396370, cg08091050, cg24131359, cg25757820, cg17331920, cg23911433, cg09694051, cg13633625, cg17078883, cg17322118, cg07079231, cg01632240, cg13785123, cg02969426, cg17567562, cg15457217, cg15169266, cg07052231, cg16102040, cg11525409, cg13618969, cg00591421, cg09166556, cg06494464, cg07804973, cg25693317, cg13589463, cg21772826, cg11809958, cg02279108, cg06784232, cg05492904, cg14523804, cg14249174, cg05006942, cg18619616, cg25539505, cg09948350, cg27466532, cg17547928, cg10480329, cg18066690, cg02244028, cg22675702, cg26872907, cg23244761, cg16636767, cg10624395, cg05923857, cg03963853, cg04851465, cg21459713, cg05242065, cg08908131, cg27366072, cg19301114, cg24788483, cg07002540, cg26124526, cg01041239, cg18589858, cg07125725, cg12655112, cg16356013, cg25376491, cg25898577, cg20641531, cg14043316, cg05452742, cg16728323, cg08694574, cg23719713, cg00646216, cg13290254, cg25517015, cg16766036, cg20918393, cg25291653, cg07505901, cg03567939, cg25765315, cg14872074, cg18770149, cg23391288, cg21983607, cg12037947, cg26953469, cg01024962, cg02780988, and cg14428166.
7. The method of item 6, including amplifying the genomic DNA of the blood specimen by a polymerase chain reaction.
8. The method of item 7, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.
9. The method of item 8, including using a regression algorithm to correlate the unmethylated CG loci observed in (b) with the unmethylated GC loci observed in the reference group of human individuals.
10. The method of item 6, wherein the observing of the unmethylated CG loci, in the genomic DNA of the human subject includes hybridizing the genomic DNA to a beadchip to create a genomic DNA hybridized bead chip and then imaging the genomic DNA hybridized beadchip
11. A method of determining percentage of blood cell subtypes in a blood specimen, comprising:
   (a) obtaining genomic DNA from the blood specimen of a human subject;
   (b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil;
   (c) comparing the unmethylated CG loci observed in (b) to unmethylated CG loci observed in genomic DNA collected from a reference group of human individuals; and
   (d) correlating the unmethylated CG loci observed in (b) with the unmethylated CG loci observed in the reference group of human individuals to determine the percentage of the blood cell subtypes in the blood specimen wherein the CG loci are cg13745692, cg15667493, cg21936454, cg15163862, cg19111287, cg03132824, cg09119854, cg04887735, cg08486432, cg18578898, cg16375820, cg11523325, cg24160371, cg05931551, cg03888645, cg11840035, cg21747310, cg07728865, cg19084822, cg21081729, cg12087639, cg24791846, cg02329886, cg11294011, cg23639055, cg25422758, cg09622285, cg26673070, cg04988216, cg09940675, cg15192986, cg23033014, cg14030593, cg26676468, cg25437005, cg19811231, cg26304744, cg27572854, cg27359629, cg08012294, cg11887733, cg12047375, cg23777479, cg15149205, cg07627628, cg24379742, cg00944599, cg15001636, cg13680246, cg12761472, cg00843117, cg17964575, cg26950756, cg07880190, cg26129027, cg15683542, cg10583651, cg18395354, cg26960322, cg10287321, cg00601124, cg04923194, cg14733637, cg14495399, cg03653880, cg24128943, cg26510178, cg20976977, cg25099065, cg07076850, cg05484949, cg13429555, cg07373578, cg13918228, cg17634650, cg00476430, cg02522485, cg19776833, cg05816398, cg02641941, cg26330304, cg27436324, cg25735823, cg10809134, cg05501327, cg22572158, cg23516026, cg25844471, cg08445080, cg15921515, cg03922997, cg15033776, cg07003611, cg16255804, cg03833097, cg05106785, cg10208609, cg02033323, cg00052246, cg02148547, cg20063728, cg20652042, cg05892024, cg25918166, cg16613029, cg13452923, cg11249120, cg04467549, cg01850245, cg04705866, cg24240349, cg12695059, cg05982017, cg09856271, cg22755579, cg05689028, cg05471842, cg00639010, cg03501128, cg04657000, cg24422171, cg10587583, cg05478809, cg22556056, cg07702659, cg17351376, cg07707056, cg20697519, cg04394724, cg00644351, cg13693582, cg23172664, cg16596052, cg14027234, cg01002223, cg16253157, cg12043508, cg16704246, cg12152193, cg06419846, cg19792099, cg14110920, cg20560862, cg07756788, cg25235205, cg19764973, cg17960717, cg00006459, cg11668148, cg09596645, cg22106847, cg07033790, cg02803925, cg14084609, cg16909341, cg05736642, cg13531735, cg12910830, cg15090899, cg24732344, cg08077807, cg00637085, cg26156120, cg27274718, cg05875239, cg13617280, cg24410690, cg05355684, cg26275360, cg08326410, cg15956469, cg27040968, cg10206039, cg09304968, cg25386954, cg03368758, cg14058851, cg01416168, cg03538296, cg17211761, cg21180201, cg21275838, cg16360310, cg24137511, cg16316183, cg25600606, cg13468144, cg23338668, cg22400420, cg10934870, cg01923724, cg04036920, cg01699630, cg25790531, cg19619956, cg26396370, cg08091050, cg24131359, cg25757820, cg17331920, cg23911433, cg10480329, cg18066690, cg02244028, cg22675702, cg26872907, cg23244761, cg16636767, cg10624395, cg05923857, cg03963853, and cg04851465.
12. The method of item 11, including amplifying the genomic DNA of the blood specimen by a polymerase chain reaction.
13. The method of item 12, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.
14. The method of item 13, including using a regression algorithm to correlate the unmethylated CG loci observed in (b) with the unmethylated GC loci observed in the reference group of human individuals.
15. The method of item 1, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a blood specimen”, as used herein, may also refer to, and encompass, a plurality of blood specimens.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ±10% of the stated numerical value.

Although the method of this disclosure has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed:

1. A method of determining percentages of twelve blood cell subtypes in a blood specimen, comprising:
(a) obtaining genomic DNA from the blood specimen of a human subject;
(b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil;
(c) comparing the unmethylated CG loci observed in (b) to unmethylated CG loci observed in genomic DNA collected from a reference group of human individuals; and
(d) correlating the unmethylated CG loci observed in (b) with the unmethylated CG loci observed in the reference group of human individuals to determine the percentage of the twelve blood cell subtypes in the blood specimen wherein the CG loci are cg21782213, cg10168351, cg03156020, cg17537714, cg13745692, cg04200580, cg15667493, cg21936454, cg15163862, cg19792189, cg10161198, cg19111287, cg05676874, cg03132824, cg17518643, cg09119854, cg11778706, cg04887735, cg22324641, cg08842268, cg09438865, cg08486432, cg04625250, cg08452021, cg20213624, cg02690807, cg22344918, cg18578898, cg19311567, cg16375820, cg08662013, cg11523325, cg21092682, cg23990235, cg24160371, cg01778017, cg05931551, cg03888645, cg21664107, cg06338239, cg01999608, cg08483099, cg23705316, cg11037382, cg11840035, cg21747310, cg20290061, cg07728865, cg19084822, cg03340102, cg03370360, cg04763150, cg11892665, cg21081729, cg24796422, cg05847670, cg00597139, cg12087639, cg24791846, cg01453386, cg14924828, cg17890852, cg07896398, cg00351957, cg25340205, cg02329886, cg21542081, cg06658453, cg08765214, cg11294011, cg03227316, cg23639055, cg25422758, cg04500050, cg15116227, cg09629758, cg02863748, cg03759549, cg25269402, cg08446606, cg19188904, cg09622285, cg26673070, cg04988216, cg21585800, cg09940675, cg14257017, cg15192986, cg14940779, cg17581877, cg11985063, cg05690666, cg20412398, cg19867897, cg23033014, cg03445419, cg17301153, cg04293820, cg14030593, cg26676468, cg03984633, cg25437005, cg19811231, cg26304744, cg27572854, cg22619531, cg09031281, cg00783080, cg00949794, cg09766370, cg27359629, cg05401393, cg08012294, cg04062032, cg11887733, cg03252427, cg12497611, cg06527889, cg13641221, cg12733242, cg18409707, cg02038353, cg12047375, cg03565732, cg18001026, cg23777479, cg15149205, cg22936341, cg07627628, cg04383721, cg05596912, cg02501773, cg15569739, cg24379742, cg00944599, cg15001636, cg13680246, cg12761472, cg19436514, cg00843117, cg17964575, cg26950756, cg27025274, cg16461337, cg13539891, cg23853980, cg07880190, cg26129027, cg15683542, cg10583651, cg05785919, cg22784669, cg18811564, cg03370491, cg18395354, cg11963313, cg26960322, cg10287321, cg27195778, cg04987479, cg02198280, cg09488807, cg07084675, cg00601124, cg27168830, cg25788647, cg02011162, cg22206842, cg20913547, cg24800373, cg15870556, cg04923194, cg14733637, cg17514643, cg03517570, cg07008893, cg14495399, cg03578212, cg26058722, cg23063536, cg19132501, cg03653880, cg24128943, cg10751207, cg10164437, cg12270000, cg07610771, cg27546582, cg00322928, cg26510178, cg21581865, cg05022030, cg22574467, cg01389234, cg26687322, cg19606289, cg06264696, cg20976977, cg14511575, cg05294459, cg25099065, cg14668581, cg10478600, cg15683107, cg07076850, cg15817752, cg07526731, cg05484949, cg13429555, cg26676273, cg06016162, cg02331158, cg16054240, cg26264767, cg08437936, cg07094228, cg27180005, cg07373578, cg13918228, cg02566312, cg09681537, cg17634650, cg25605180, cg00476430, cg18358155, cg01841368, cg04496578, cg02522485, cg16495530, cg07390370, cg25904998, cg05810879, cg24367295, cg19776833, cg14095525, cg16289232, cg05816398, cg02641941, cg26330304, cg04429341, cg11518040, cg03748473, cg04982372, cg27436324, cg06681449, cg25735823, cg06738507, cg10809134, cg11812782, cg25271915, cg05501327, cg22572158, cg04003505, cg22438885, cg12882261, cg23516026, cg23148481, cg25844471, cg10419892, cg15463664, cg08445080, cg15921515, cg24306984, cg23539484, cg03922997, cg05487307, cg27340845, cg03217619, cg13477627, cg15033776, cg15164173, cg08358686, cg08888548, cg00678912, cg15560912, cg07003611, cg16255804, cg07833643, cg26451108, cg08337454, cg15908108, cg17642929, cg03833097, cg18857045, cg23411838, cg00409021, cg17290867, cg16044562, cg02105817, cg18459351, cg05106785, cg10208609, cg16901161, cg02033323, cg24998002, cg07461115, cg04311057, cg06026449, cg00052246, cg02148547, cg02203103, cg20063728, cg20813776, cg13475329, cg13211380, cg20652042, cg10651616, cg02826841, cg05892024, cg25918166, cg03267512, cg27298253, cg18327204, cg19653890, cg02319946, cg18073938, cg21023071, cg07661702, cg16613029, cg13452923, cg11749308, cg10220401, cg08657911, cg17523148, cg24898812, cg04017239, cg22250168, cg11249120, cg04467549, cg01850245, cg11693925, cg04705866, cg24240349, cg01923140, cg12695059, cg05036909, cg05982017, cg26429965, cg09856271, cg08243981, cg14860676, cg22755579, cg05689028, cg15661018, cg05471842, cg00639010, cg19253785, cg16438469, cg03501128, cg04657000, cg15213052, cg24422171, cg19850895, cg10587583, cg05478809, cg25448766, cg22556056, cg03077364, cg18329839, cg07702659, cg17351376, cg13487687, cg07707056, cg20697519, cg04394724, cg12742224, cg00644351, cg13693582, cg23172664, cg16596052, cg14027234, cg01002223, cg04928784, cg16253157, cg12043508, cg16704246, cg04047276, cg16070350, cg20330224, cg02053685, cg00790439, cg26796793, cg20492070, cg12152193, cg16780406, cg06419846, cg18844901, cg19792099, cg14110920, cg21631387, cg20560862, cg17445453, cg02865554, cg07756788, cg09799856, cg17829179, cg25235205, cg11020586, cg20587232, cg04249416, cg19764973, cg24818060, cg06421734, cg11649969, cg04148640, cg01355027, cg15301710, cg01511347, cg17960717, cg00006459, cg15873326, cg11668148, cg09596645, cg08538069, cg02233915, cg22106847, cg07033790, cg05840984, cg10220104, cg17030380, cg02803925, cg14084609, cg16909341, cg05736642, cg13531735, cg01404879, cg21503771, cg15662508, cg05906696, cg09803743, cg12910830, cg15090899, cg22034357, cg17105176, cg24732344, cg15997319, cg09010228, cg11262246, cg05089849, cg22954836, cg02179849, cg25293601, cg24057263, cg01048814, cg08077807, cg00637085, cg17542165, cg13123707, cg26156120, cg18852698, cg18629096, cg27274718, cg05875239, cg13617280, cg24410690, cg05355684, cg18584272, cg18546155, cg26275360, cg14563201, cg08326410, cg02855870, cg15956469, cg27040968, cg10206039, cg09932794, cg09304968, cg00949861, cg02847139, cg25386954, cg04737115, cg03368758, cg14058851, cg23369683, cg14047092, cg27495654, cg22829007, cg16995933, cg01416168, cg08530578, cg07543798, cg12851731, cg15646529, cg03538296, cg13257250, cg17211761, cg18948661, cg13598219, cg22668643, cg09134152, cg21180201, cg08907176, cg03559700, cg21275838, cg05508074, cg18857340, cg12973514, cg16360310, cg24137511, cg16316183, cg25600606, cg18646784, cg05956380, cg16683571, cg18330536, cg17781418, cg13468144, cg26849760, cg23338668, cg13155300, cg24392753, cg22400420, cg10934870, cg14095610, cg01923724, cg06500418, cg04179951, cg16597887, cg04036920, cg15715903, cg01701207, cg08218512, cg07619820, cg01699630, cg25790531, cg19619956, cg26396370, cg05170732, cg08091050, cg09622624, cg02379348, cg25576801, cg27393431, cg09601944, cg24131359, cg26979142, cg04682832, cg25757820, cg26789411, cg17331920, cg00434123, cg16107565, cg23911433, cg12578084, cg15810023, cg08771884, cg07993518, cg20030158, cg20077668, cg27646850, cg15173892, cg07617198, cg10480329, cg20374292, cg00771778, cg18066690, cg02647842, cg20290017, cg02244028, cg06636137, cg25605773, cg22675702, cg08844422, cg26872907, cg23244761, cg23373670, cg14172797, cg26201751, cg02290460, cg18356337, cg00908794, cg08965078, cg16636767, cg09592164, cg17195809, cg19336573, cg03135975, cg05065075, cg01407558, cg08097676, cg23312369, cg11620863, cg10624395, cg03556458, cg14116399, cg19501306, cg02007480, cg23950707, cg14458725, cg14898005, cg01950767, cg18844042, cg05923857, cg01842756, cg03963853, cg11183587, cg21185441, cg02647748, cg14806960, and cg04851465.

2. The method of claim 1, including amplifying the genomic DNA of the blood specimen by a polymerase chain reaction.

3. The method of claim 2, wherein the observing of the unmethylated CG loci, in the genomic DNA of the human subject includes hybridizing the genomic DNA to a beadchip to create a genomic DNA hybridized beadchip and then imaging the genomic DNA hybridized beadchip.

4. The method of claim 3, including using a regression algorithm to correlate the unmethylated CG loci observed in (b) with the unmethylated GC loci observed in the reference group of human individuals.

5. The method of claim 1, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.

6. A method of determining percentage of twelve blood cell subtypes in a blood specimen, comprising:
(a) obtaining genomic DNA from the blood specimen of a human subject;
(b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil;
(c) comparing the unmethylated CG loci observed in (b) to unmethylated CG loci observed in genomic DNA collected from a reference group of human individuals; and
(d) correlating the unmethylated CG loci observed in (b) with the unmethylated CG loci observed in the reference group of human individuals to determine the percentage of the twelve blood cell subtypes in the blood specimen wherein the CG loci are cg13745692, cg15667493, cg21936454, cg15163862, cg19111287, cg03132824, cg09119854, cg04887735, cg08486432, cg18578898, cg16375820, cg11523325, cg24160371, cg05931551, cg03888645, cg11840035, cg21747310, cg07728865, cg19084822, cg17894779, cg24503957, cg14138047, cg00694660, cg13484324, cg01364581, cg02725065, cg07133479, cg02280893, cg03069943, cg07643762, cg21200667, cg06828418, cg17824906, cg00378228, cg24463664, cg18984002, cg10091155, cg09177577, cg11840104, cg15958839, cg16192197, cg13104185, cg15009294, cg00629382, cg00705661, cg00208274, cg24211304, cg20558320, cg04073934, cg05158109, cg21081729, cg12087639, cg24791846, cg02329886, cg11294011, cg23639055, cg25422758, cg09622285, cg26673070, cg04988216, cg09940675, cg15192986, cg23033014, cg14030593, cg26676468, cg19699264, cg17170133, cg12037509, cg03380342, cg24736010, cg17436460, cg18469148, cg24496563, cg21039188, cg05506809, cg00712106, cg12646067, cg03184452, cg07862744, cg14633252, cg17282428, cg02587120, cg00551398, cg26009797, cg00086283, cg09275783, cg15106420, cg22197708, cg15003763, cg01782059, cg13894852, cg09131332, cg07188551, cg26252958, cg06706029, cg13394713, cg26603598, cg05201777, cg11809342, cg23743708, cg25437005, cg19811231, cg26304744, cg27572854, cg27359629, cg08012294, cg11887733, cg12047375, cg23777479, cg15149205, cg07627628, cg24379742, cg00944599, cg15001636, cg13680246, cg12761472, cg00843117, cg17964575, cg26950756, cg07880190, cg26129027, cg15683542, cg10583651, cg18289508, cg23005797, cg07784293, cg09222732, cg16497398, cg02619107, cg12419766, cg07340499, cg06828613, cg23635663, cg23506728, cg27297043, cg24525744, cg06288253, cg25404597, cg21814995, cg00292305, cg19622138, cg18303608, cg27518047, cg18835815, cg18051361, cg10295609, cg05519606, cg23271530, cg13011976, cg09449150, cg18395354, cg26960322, cg10287321, cg00601124, cg04923194, cg14733637, cg14495399, cg03653880, cg24128943, cg26510178, cg20976977, cg07316730, cg00148025, cg25382573, cg15829423, cg08220696, cg26383838, cg22084515, cg07960360, cg09241714, cg08761324, cg01884715, cg07044414, cg03680932, cg06369076, cg04330057, cg00083596, cg27576271, cg13678009, cg14101687, cg20561261, cg27157619, cg23900696, cg12208691, cg18822703, cg04329659, cg13551330, cg03822267, cg27416848, cg00087511, cg19277389, cg03717570, cg06099459, cg06541733, cg23731501, cg02079700, cg06419212, cg18861762, cg27317694, cg06013895, cg25099065, cg07076850, cg05484949, cg13429555, cg07373578, cg13918228, cg17634650, cg00476430, cg02522485, cg19776833, cg05816398, cg02641941, cg26330304, cg27436324, cg25735823, cg10809134, cg27184438, cg10361659, cg03653817, cg03328673, cg17453767, cg08246318, cg00649632, cg13276502, cg20847766, cg03386536, cg01598421, cg14422093, cg16098726, cg03238899, cg08986727, cg00374717, cg00863309, cg10678215, cg14905514, cg15552491, cg04074908, cg25107522, cg07433769, cg16586807, cg02563364, cg11972721, cg12796332, cg22528123, cg16417374, cg23325384, cg06916059, cg09299082, cg05776861, cg01502811, cg05501327, cg22572158, cg23516026, cg25844471, cg08445080, cg15921515, cg03922997, cg15033776, cg07003611, cg16255804, cg03833097, cg05106785, cg10208609, cg02033323, cg00052246, cg02148547, cg26408382, cg08562111, cg22360028, cg09490124, cg09197075, cg18124517, cg05878535, cg23519969, cg17498667, cg00463840, cg09642378, cg02397552, cg19876232, cg12485044, cg13345122, cg17392230, cg01554304, cg25557227, cg23541811, cg03873220, cg16685586, cg02979010, cg06840801, cg01865118, cg01436550, cg11812071, cg16637690, cg22980427, cg22577252, cg07805771, cg24599709, cg02272851, cg24847035, cg07793163, cg20063728, cg20652042, cg05892024, cg25918166, cg16613029, cg13452923, cg11249120, cg04467549, cg01850245, cg04705866, cg24240349, cg12695059, cg05982017, cg09856271, cg22755579, cg05689028, cg05471842, cg00639010, cg03501128, cg04657000, cg24037746, cg00460916, cg24618739, cg09736286, cg12983578, cg20417024, cg23320693, cg13805183, cg08035195, cg09096234, cg11161150, cg17542594, cg10335493, cg14776168, cg02763540, cg16946840, cg03315940, cg01356867, cg02586198, cg25503323, cg12524168, cg26509305, cg07109551, cg02205013, cg19884600, cg09088625, cg03534847, cg08877257, cg12159213, cg05905180, cg24422171, cg10587583, cg05478809, cg22556056, cg07702659, cg17351376, cg07707056, cg20697519, cg04394724, cg00644351, cg13693582, cg23172664, cg16596052, cg14027234, cg01002223, cg16253157, cg12043508, cg16704246, cg12152193, cg06419846, cg19792099, cg14110920, cg20560862, cg07756788, cg25235205, cg01949403, cg14507427, cg05496363, cg09872088, cg23463186, cg14468508, cg20367218, cg12221970, cg26856443, cg09628822, cg12655059, cg08585614, cg26668000, cg16359142, cg11067179, cg09127592, cg10365743, cg19556572, cg09586583, cg22213242, cg03788408, cg06748978, cg26354221, cg14537031, cg20681184, cg19764973, cg17960717, cg00006459, cg11668148, cg09596645, cg22106847, cg07033790, cg02803925, cg14084609, cg16909341, cg05736642, cg13531735, cg12910830, cg15090899, cg24732344, cg08077807, cg00637085, cg26156120, cg21237481, cg23153546, cg25203627, cg18550847, cg04538585, cg06680065, cg00391067, cg11197258, cg16362140, cg06315149, cg00170714, cg07410597, cg08061334, cg14209186, cg25578728, cg14697334, cg27000590, cg25381747, cg24706564, cg20315954, cg07766916, cg17732216, cg19788934, cg08969102, cg15427040, cg17738684, cg19968199, cg24459209, cg20240243, cg16472853, cg07201450, cg01820584, cg27274718, cg05875239, cg13617280, cg24410690, cg05355684, cg26275360, cg08326410, cg15956469, cg27040968, cg10206039, cg09304968, cg25386954, cg03368758, cg14058851, cg01416168, cg03538296, cg17211761, cg21180201, cg21275838, cg16360310, cg24137511, cg16316183, cg04992852, cg25846372, cg07802362, cg13254203, cg01112784, cg05253716, cg15705930, cg18087143, cg23001370, cg07873128, cg10830795, cg03891962, cg09278623, cg03055021, cg25029657, cg17169982, cg10157558, cg00515905, cg23617121, cg27309871, cg21777154, cg07123481, cg22917487, cg16220743, cg03956353, cg04718702, cg01830256, cg11790417, cg25600606, cg13468144, cg23338668, cg22400420, cg10934870, cg01923724, cg04036920, cg01699630, cg25790531, cg19619956, cg26396370, cg08091050, cg24131359, cg25757820, cg17331920, cg23911433, cg09694051, cg13633625, cg17078883, cg17322118, cg07079231, cg01632240, cg13785123, cg02969426, cg17567562, cg15457217, cg15169266, cg07052231, cg16102040, cg11525409, cg13618969, cg00591421, cg09166556, cg06494464, cg07804973, cg25693317, cg13589463, cg21772826, cg11809958, cg02279108, cg06784232, cg05492904, cg14523804, cg14249174, cg05006942, cg18619616, cg25539505, cg09948350, cg27466532, cg17547928, cg10480329, cg18066690, cg02244028, cg22675702, cg26872907, cg23244761, cg16636767, cg10624395, cg05923857, cg03963853, cg04851465, cg21459713, cg05242065, cg08908131, cg27366072, cg19301114, cg24788483, cg07002540, cg26124526, cg01041239, cg18589858, cg07125725, cg12655112, cg16356013, cg25376491, cg25898577, cg20641531, cg14043316, cg05452742, cg16728323, cg08694574, cg23719713, cg00646216, cg13290254, cg25517015, cg16766036, cg20918393, cg25291653, cg07505901, cg03567939, cg25765315, cg14872074, cg18770149, cg23391288, cg21983607, cg12037947, cg26953469, cg01024962, cg02780988, and cg14428166.

7. The method of claim 6, including amplifying the genomic DNA of the blood specimen by a polymerase chain reaction.

8. The method of claim 7, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.

9. The method of claim 8, including using a regression algorithm to correlate the unmethylated CG loci observed in (b) with the unmethylated GC loci observed in the reference group of human individuals.

10. The method of claim 6, wherein the observing of the unmethylated CG loci, in the genomic DNA of the human subject includes hybridizing the genomic DNA to a beadchip to create a genomic DNA hybridized bead chip and then imaging the genomic DNA hybridized beadchip 11. A method of determining percentage of blood cell subtypes in a blood specimen, comprising:
(a) obtaining genomic DNA from the blood specimen of a human subject;
(b) observing unmethylated CG loci, in the genomic DNA of the human subject, wherein said observing includes performing a bisulfate conversion process on the genomic DNA of the human subject so that cytosine residues in the genomic DNA of the human subject are transformed to uracil, while 5-methylcytosine residues in the genomic DNA of the human subject are not transformed to uracil;
(c) comparing the unmethylated CG loci observed in (b) to unmethylated CG loci observed in genomic DNA collected from a reference group of human individuals; and
(d) correlating the unmethylated CG loci observed in (b) with the unmethylated CG loci observed in the reference group of human individuals to determine the percentage of the blood cell subtypes in the blood specimen wherein the CG loci are cg13745692, cg15667493, cg21936454, cg15163862, cg19111287, cg03132824, cg09119854, cg04887735, cg08486432, cg18578898, cg16375820, cg11523325, cg24160371, cg05931551, cg03888645, cg11840035, cg21747310, cg07728865, cg19084822, cg21081729, cg12087639, cg24791846, cg02329886, cg11294011, cg23639055, cg25422758, cg09622285, cg26673070, cg04988216, cg09940675, cg15192986, cg23033014, cg14030593, cg26676468, cg25437005, cg19811231, cg26304744, cg27572854, cg27359629, cg08012294, cg11887733, cg12047375, cg23777479, cg15149205, cg07627628, cg24379742, cg00944599, cg15001636, cg13680246, cg12761472, cg00843117, cg17964575, cg26950756, cg07880190, cg26129027, cg15683542, cg10583651, cg18395354, cg26960322, cg10287321, cg00601124, cg04923194, cg14733637, cg14495399, cg03653880, cg24128943, cg26510178, cg20976977, cg25099065, cg07076850, cg05484949, cg13429555, cg07373578, cg13918228, cg17634650, cg00476430, cg02522485, cg19776833, cg05816398, cg02641941, cg26330304, cg27436324, cg25735823, cg10809134, cg05501327, cg22572158, cg23516026, cg25844471, cg08445080, cg15921515, cg03922997, cg15033776, cg07003611, cg16255804, cg03833097, cg05106785, cg10208609, cg02033323, cg00052246, cg02148547, cg20063728, cg20652042, cg05892024, cg25918166, cg16613029, cg13452923, cg11249120, cg04467549, cg01850245, cg04705866, cg24240349, cg12695059, cg05982017, cg09856271, cg22755579, cg05689028, cg05471842, cg00639010, cg03501128, cg04657000, cg24422171, cg10587583, cg05478809, cg22556056, cg07702659, cg17351376, cg07707056, cg20697519, cg04394724, cg00644351, cg13693582, cg23172664, cg16596052, cg14027234, cg01002223, cg16253157, cg12043508, cg16704246, cg12152193, cg06419846, cg19792099, cg14110920, cg20560862, cg07756788, cg25235205, cg19764973, cg17960717, cg00006459, cg11668148, cg09596645, cg22106847, cg07033790, cg02803925, cg14084609, cg16909341, cg05736642, cg13531735, cg12910830, cg15090899, cg24732344, cg08077807, cg00637085, cg26156120, cg27274718, cg05875239, cg13617280, cg24410690, cg05355684, cg26275360, cg08326410, cg15956469, cg27040968, cg10206039, cg09304968, cg25386954, cg03368758, cg14058851, cg01416168, cg03538296, cg17211761, cg21180201, cg21275838, cg16360310, cg24137511, cg16316183, cg25600606, cg13468144, cg23338668, cg22400420, cg10934870, cg01923724, cg04036920, cg01699630, cg25790531, cg19619956, cg26396370, cg08091050, cg24131359, cg25757820, cg17331920, cg23911433, cg10480329, cg18066690, cg02244028, cg22675702, cg26872907, cg23244761, cg16636767, cg10624395, cg05923857, cg03963853, and cg04851465.

12. The method of claim 11, including amplifying the genomic DNA of the blood specimen by a polymerase chain reaction.

13. The method of claim 12, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.

14. The method of claim 13, including using a regression algorithm to correlate the unmethylated CG loci observed in (b) with the unmethylated GC loci observed in the reference group of human individuals.

15. The method of claim 1, wherein the observing of the unmethylated CG loci, in the genomic DNA of the human subject includes hybridizing the genomic DNA to a beadchip to create a genomic DNA hybridized bead chip and then imaging the genomic DNA hybridized beadchip.
